(19) United States
(12) Patent Application Publication
(10) Pub. No.: US 2005/0182073 A1
(43) Pub. Date: Aug. 18, 2005

(54) 5-Phenylpyrimidines

(76) Inventors: Olaf Gebauer, Koln (DE); Herbert Gayer, Monheim (DE); Ulrich Heinemann, Leichlingen (DE); Stefan Herrmann, Langenfeld (DE); Stefan Hillebrand, Neuss (DE); Ronald Ebbert, Monheim (DE); Peter Dahmen, Neuss (DE); Karl-Heinz Kuck, Langenfeld (DE); Ulrike Wachendorff-Neumann, Neuwied (DE)

Correspondence Address: BAYER CROPSCIENCE LP;Patent Department, 100 BAYER ROAD, PITTSBURGH, PA, 15205-9741, US

(21) Appl. No.: 11/039,273

(22) Filed: Jan. 20, 2005

(30) Foreign Application Priority Data
Jan. 23, 2004 (DE) 1020040034931

(51) Int. Cl. A01N 43/78; C07D 43/02

(52) U.S. Cl. 514/256; 544/333; 544/296

(57) ABSTRACT

The present invention relates to novel 5-phenylpyrimidines, to a plurality of processes for their preparation, and to their use for controlling unwanted microorganisms. The invention further relates to novel intermediates and to processes for their preparation.

The present invention relates to novel 5-phenylpyrimidines, to a plurality of processes for their preparation and to their use for controlling unwanted microorganisms. Moreover, the invention relates to novel intermediates and to processes for their preparation.

It is already known that certain 5-phenylpyrimidines have fungicidal properties (cf. WO 03/070721, WO 02/074753, WO 01/96314, WO 03/43993). The activity of these compounds is good; however, at low application rates it is sometimes unsatisfactory.

Accordingly, it was an object of the present invention to provide further compounds having good activity against microorganisms, in particular fungicidal activity.

We have now found novel 5-phenylpyrimidines of the formula 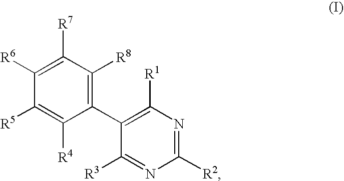
in which
R¹ represents C₁-C₈-alkyl, C₂-C₈-alkenyl, C₂-C₈-alkynyl, C₃-C₈-cycloalkyl, C₃-C₈-cycloalkenyl or represents a three- to ten-membered saturated mono- or bicyclic heterocycle which is attached via carbon and contains one to four heteroatoms from the group consisting of O, N and S, where R¹ may be substituted by one to three identical or different groups R^{a} and
   R^{a} represents halogen, alkylamino, alkylhydrazino, cyano, oxo, nitro, C₁-C₆-alkylthio, C₁-C₆-alkyl, C₁-C₆-haloalkyl, tri(C₁-C₄-alkyl)silyl and/or C₁-C₆-alkoxy and/or represents unsubstituted or halogen-, C₁-C₄-alkyl- or C₁-C₄-haloalkyl-substituted C₃-C₆-cycloalkyl,
R² represents a three- to ten-membered saturated, partially unsaturated or aromatic mono- or bicyclic heterocycle which contains one to four heteroatoms from the group consisting of O, N and S, where R² may be substituted by one to three identical or different groups R^{b}, and
   R^{b} represents halogen, hydroxyl, cyano, oxo, nitro, amino, mercapto, C₁-C₆-alkyl, C₁-C₆-haloalkyl, C₂-C₆-alkenyl, C₂-C₆-alkynyl, C₃-C₆-cycloalkyl, C₁-C₆-alkoxy, C₁-C₆-haloalkoxy, carboxyl, C₁-C₇-alkoxycarbonyl, carbamoyl, C₁-C₇-alkylaminocarbonyl, C₁-C₆-alkyl-C₁-C₆-alkylaminocarbonyl, morpholinocarbonyl, pyrrolidinocarbonyl, C₁-C₇-alkylcarbonylamino, C₁-C₆-alkylamino, di-(C₁-C₆-alkyl)amino, C₁-C₆-alkylthio, C₁-C₆-alkylsulphinyl, C₁-C₆-alkylsulphonyl, hydroxysulphonyl, aminosulphonyl, C₁-C₆-alkylaminosulphonyl and/or di-(C₁-C₆-alkyl)aminosulphonyl;
R³ represents halogen or C₁-C₈-alkyl, C₁-C₈-alkoxy, C₁-C₈-haloalkyl, C₁-C₈-alkylthio, C₁-C₈-alkylsulphinyl, C₁-C₈-alkylsulphonyl, or cyano,
R⁴, R⁸ independently of one another represent hydrogen, halogen, C₁-C₆-alkyl, C₁-C₆-haloalkyl or C₁-C₆-alkoxy,
R⁵, R⁷ independently of one another represent hydrogen, halogen, C₁-C₆-alkyl, C₁-C₆-haloalkyl, unsubstituted or halogen- or C₁-C₆-alkyl-substituted C₃-C₆-cycloalkyl, phenyl, phenoxy or phenylthio, where R⁵ and R⁷, when they represent phenyl, phenoxy or phenylthio, may be substituted by one to three identical or different groups R^{c}, and
   R^{c} represents halogen, cyano, nitro, C₁-C₆-alkyl, C₁-C₆-haloalkyl, C₁-C₆-alkoxy and/or C₃-C₆-cycloalkyl,
R⁶ represents hydrogen, halogen, cyano, nitro, C₁-C₆-alkyl, C₁-C₆-haloalkyl, C₁-C₆-alkoxy, C₃-C₆-cycloalkoxy, C₁-C₆-haloalkoxy, C₁-C₆-alkoxycarbonyl, C₁-C₆-haloalkoxycarbonyl, unsubstituted or halogen- or C₁-C₆-alkyl-substituted C₃-C₆-cycloalkyl, aminocarbonyl, C₁-C₆-alkylcarbonyl, C₁-C₆-alkylaminocarbonyl, C₁-C₆-dialkylaminocarbonyl, C₁-C₆-alkylthio; C₁-C₆-alkylsulphinyl; C₁-C₆-alkylsulphonyl, alkylaminosulphonyl or dialkylaminosulphonyl or phenyl, phenoxy, phenylthio, where R⁶, when it represents phenyl, phenoxy or phenylthio, may be substituted by one to three identical or different groups R^{d} and
   R^{d} represents halogen, cyano, nitro, C₁-C₆-alkyl, C₁-C₆-haloalkyl, C₁-C₆-alkoxy and/or C₃-C₆-cycloalkyl.

Compared to the compounds known from the prior art, the compounds of the formula I have higher activity against unwanted microorganisms.

The compounds of the formula I can be obtained by different routes.

It has now been found that 5-phenylpyrimidines of the formula I in which R² represents a heterocycle which is attached via nitrogen and R³ represents halogen (compounds of the formula (I′)) can be prepared (process a)) by oxidizing compounds of the formula 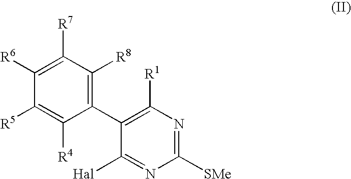
in which
R¹, R⁴, R⁵, R⁶, R⁷ and R⁸ are as defined above and Hal represents halogen, with an oxidizing agent, if appropriate in the presence of a diluent, and reacting the resulting compounds of the formula 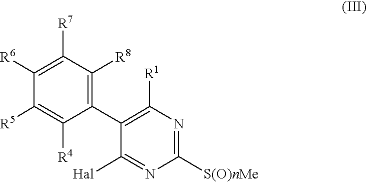
in which
R¹, R⁴, R⁵, R⁶, R⁷, R⁸ und Hal are as defined above and n=1 or 2,
   with a compound of the formula
   R²—H (IV),
in which R² is as defined above, if appropriate in the presence of a diluent and if appropriate in the presence of an acid acceptor.

5-Phenylpyrimidines of the formula I in which R² represents a heterocycle which is attached via carbon and R³ represents halogen (compounds of the formula (I″)) can be prepared (process b)) by reacting compounds of the formula 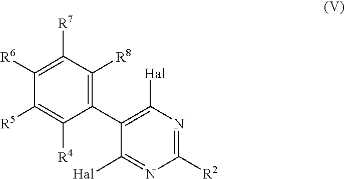
in which
R¹, R², R⁴, R⁵, R⁶, R⁷ and R⁸ are as defined above and Hal represents halogen,
   with a compound of the formula
   R¹-M¹ (VI),
in which
R¹ is as defined above and
M¹ represents lithium, sodium, potassium, dihydroxyboranyl, a radical of the formula 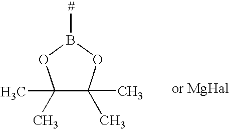
in which
Hal represents chlorine, bromine or iodine,
   if appropriate in the presence of a diluent and if appropriate in the presence of a catalyst.

5-Phenylpyrimidines of the formula I in which R² represents a heterocycle which is attached via nitrogen or carbon and R³ represents C₁-C₈-alkyl, C₁-C₈-alkoxy, C₁-C₈-alkylthio, C₁-C₈-alkylsulphinyl, C₁-C₈-alkylsulphonyl or cyano (compounds of the formula I′″) can be prepared (process c)) by reacting compounds of the formula (I′) or (I″) 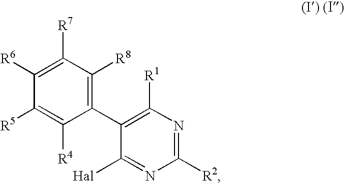
in which
R¹, R², R⁴, R⁵, R⁶, R⁷ and R⁸ are as defined above and Hal represents
halogen
   either
c1) with a compound of the formula
   R³-M² (VII),
   in which
   R³ represents C₁-C₈-alkoxy, C₁-C₈-alkylthio, C₁-C₈-alkylsulphinyl, C₁-C₈-alkylsulphonyl or cyano and
   M² represents sodium or potassium,
   if appropriate in the presence of a diluent,
      or
c2) with Grignard compounds of the formula
   R³—Mg Hal (VIII)
   in which
   R³ represents C₁-C₈-alkyl,
   Hal represents chlorine or bromine,
   in the presence of a diluent and, if appropriate, in the presence of a catalyst.

Alternatively, 5-phenylpyrimidines of the formula I (compounds of the formula (I^{iv})) in which R² represents a heterocycle which is attached via nitrogen or carbon and R³ represents C₁-C₈-alkyl or C₁-C₈-haloalkyl can be prepared (process d)) by reacting
d1) compounds of the formula 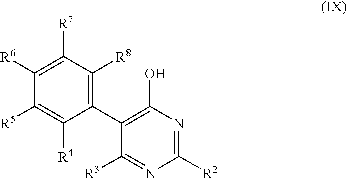
   in which
   R², R³, R⁴, R⁵, R⁶, R⁷ and R⁸ are as defined above with a halogenating agent, if appropriate in the presence of an acid acceptor and if appropriate in the presence of a diluent, and
d2) reacting the resulting compounds of the formula 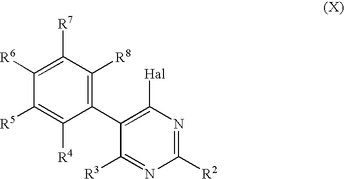
   in which
   R², R³, R⁴, R⁵, R⁶, R⁷ and R⁸ are as defined above and Hal represents
   halogen
   with a compound of the formula
      R¹-M¹ (VI),
   in which
   R¹ is as defined above and
   M¹ represents lithium, sodium, potassium, dihydroxyboranyl, a radical of the formula 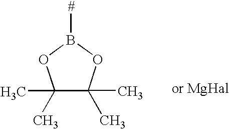
   in which
   Hal represents chlorine, bromine or iodine,
   if appropriate in the presence of a diluent and if appropriate in the presence of a catalyst.

Finally, it has been found that the 5-phenylpyrimidines of the formula (I) are highly suitable for controlling unwanted microorganisms. In particular, they have a potent fungicidal activity, and they can be employed both in crop protection and in the protection of material.

The compounds of the formula (I) according to the invention can, if appropriate, be present as mixtures of different possible isomeric forms, in particular of stereoisomers, such as E and Z, threo and erythro and also optical isomers, such as R and S isomers, or atropisomers, and, if appropriate, also of tautomers.

In the definitions of the symbols given in the formulae above, collective terms were used which are generally representative of the following substituents:
halogen: fluorine, chlorine, bromine and iodine;
alkyl: saturated straight-chain or branched hydrocarbon radicals having 1 to 4, 6 or 8 carbon atoms, for example C₁-C₆-alkyl, such as methyl, ethyl, propyl, 1-methyl-ethyl, butyl, 1-methyl-propyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;
haloalkyl: straight-chain or branched alkyl groups having 1 to 8 carbon atoms (as mentioned above), where some or all of the hydrogen atoms in this group may be replaced by halogen atoms as mentioned above, for example C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and 1,1,1-trifluoroprop-2-yl;
alkenyl: unsaturated straight-chain or branched hydrocarbon radicals having 2 to 4, 6 or 8 carbon atoms and a double bond in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, t-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
alkynyl: straight-chain or branched hydrocarbon groups having 2 to 4, 6 or 8 carbon atoms and a triple bond in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
cycloalkyl: monocyclic saturated hydrocarbon groups having 3 to 6 carbon ring members, such as cyclopropyl, cyclobutyl, cyclopentyl and cyclohexyl;
cycloalkyl: monocyclic nonaromatic hydrocarbon groups having 3 to 8 carbon ring members and at least one double bond, such as cyclopenten-1-yl, cyclohexen-1-yl, cyclohepta-1,3-dien-1-yl;
alkoxycarbonyl: an alkoxy group having 1 to 6 carbon atoms (as mentioned above) which is attached to the skeleton via a carbonyl group (—CO—);
oxyalkyleneoxy: divalent unbranched chains of 1 to 3 CH₂ groups, where both valencies are attached to the skeleton via an oxygen atom, for example OCH₂O, OCH₂CH₂O and OCH₂CH₂CH₂O;
a three- to ten-membered saturated or partially unsaturated heterocycle which contains one to four heteroatoms from the group consisting of oxygen, nitrogen and sulphur: mono- or bicyclic heterocycles (heterocyclyl) which contain, in addition to carbon ring members, one to three nitrogen atoms and/or one oxygen or sulphur atom or one or two oxygen and/or sulphur atoms; if the ring contains a plurality of oxygen atoms, these are not directly adjacent; for example oxiranyl, aziridinyl, 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydro-triazin-2-yl and 1,2,4-hexahydrotriazin-3-yl;
a five- to ten-membered aromatic heterocycle which contains one to four heteroatoms from the group consisting of oxygen, nitrogen and sulphur: mono- or bicyclic heteroaryl, for example
   5-membered heteroaryl which contains one to four nitrogen atoms or one to three nitrogen atoms and one sulphur or oxygen atom: 5-membered heteroaryl groups which, in addition to carbon atoms, may contain one to four nitrogen atom or one to three nitrogen atoms and one sulphur or oxygen atom as ring members, for example 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;
   benzo-fused 5-membered heteroaryl which contains one to three nitrogen atoms or one nitrogen atom and one oxygen or sulphur atom: 5-membered heteroaryl groups which, in addition to carbon atoms, may contain one to four nitrogen atoms or one to three nitrogen atoms and one sulphur or oxygen atom as ring members and in which two adjacent carbon ring members or one nitrogen and one adjacent carbon ring member may be bridged by a buta-1,3-diene-1,4-diyl group, in which one or two carbon atoms may be replaced by nitrogen atoms;
   5-membered heteroaryl which is attached via nitrogen and contains one to four nitrogen atoms, or benzo-fused 5-membered heteroaryl which is attached via nitrogen and contains one to three nitrogen atoms: 5-membered heteroaryl groups which, in addition to carbon atoms, may contain one to four nitrogen atoms or one to three nitrogen atoms as ring members and in which two adjacent carbon ring members or one nitrogen and one adjacent carbon ring member may be bridged by a buta-1,3-diene-1,4-diyl group in which one or two carbon atoms may be replaced by nitrogen atoms, where these rings are attached to the skeleton via one of the nitrogen ring members, for example 1-pyrrolyl, 1-pyrazolyl, 1,2,4-triazol-1-yl, 1-imidazolyl, 1,2,3-triazol-1-yl, 1,3,4-triazol-1-yl;
6-membered heteroaryl which contains one to three or one to four nitrogen atoms: 6-membered heteroaryl groups which, in addition to carbon atoms, may contain one to three or one to four nitrogen atoms as ring members, for example 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

The particularly preferred embodiments of the intermediates with respect to the variables correspond to those of the radicals R¹ to R⁸ of formula I.

For the end products of the formula (I) and also, correspondingly, for the starting materials or intermediates required in each case for the preparation, the following meanings of the substituents are particularly preferred, in each case on their own or in combination.

Preference is given to compounds of the formula I in which R¹ represents C₁-C₆-alkyl, C₂-C₆-alkenyl, C₃-C₆-alkynyl, C₃-C₆-cycloalkyl, or represents a three- to ten-membered saturated mono- or bicyclic heterocycle which is attached via carbon and contains one heteroatom from the group consisting of O, N and S, where R¹ may be substituted by one to three identical or different groups R^{a}.

Particular preference is given to compounds of the formula (I) in which R¹ is substituted by one to three identical or different of the groups R^{a} below: halogen, alkylamino, alkylhydrazino, cyano, oxo, nitro, C₁-C₄-alkylthio, C₁-C₄-alkyl, C₁-C₄-haloalkyl, trimethylsilyl and/or C₁-C₄-alkoxy and/or unsubstituted or halogen-, C₁-C₄-alkyl- or C₁-C₄-haloalkyl-substituted C₃-C₆-cycloalkyl.

Particularly preferred substituents R^{a} are halogen, C₁-C₄-alkyl, C₁-C₄-alkoxy or cyano.

Very particular preference is given to compounds of the formula (I) in which R¹ is unsubstituted or monosubstituted by halogen, cyanol, methyl, ethyl, methoxy, ethoxy or trifluoromethyl.

Moreover, very particular preference is given to compounds of the formula I

in which

R¹ represents a radical of the formula 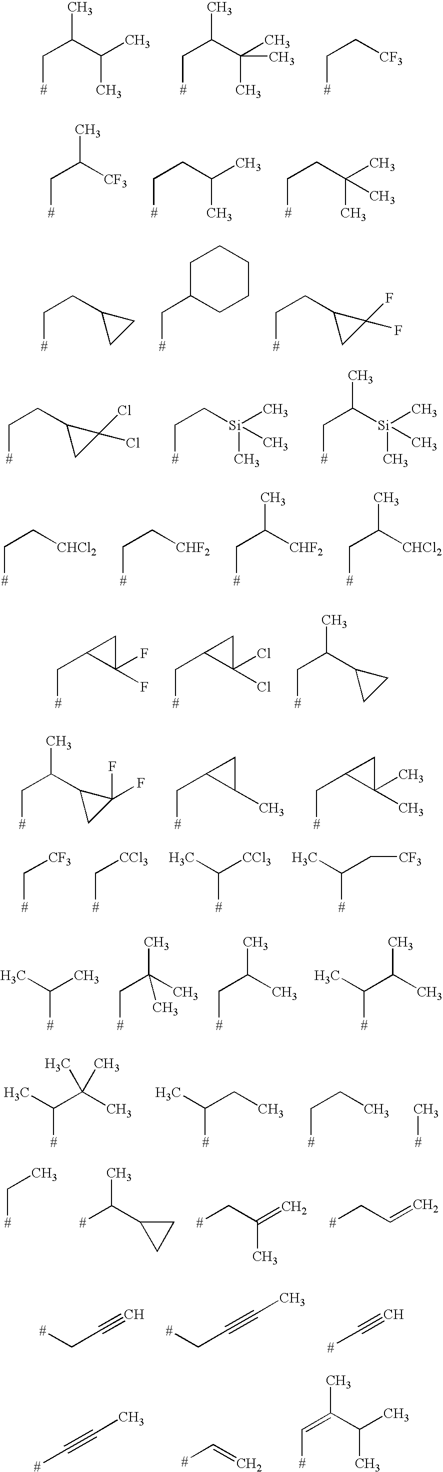 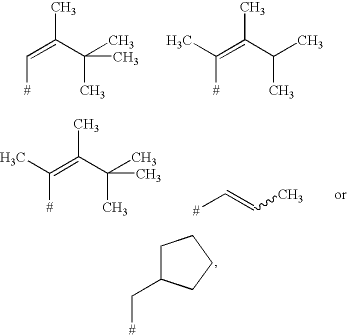
   where # in each case marks the point of attachment.

Very particular preference is likewise given to compounds of the formula I in which
R¹ represents a radical of the formula 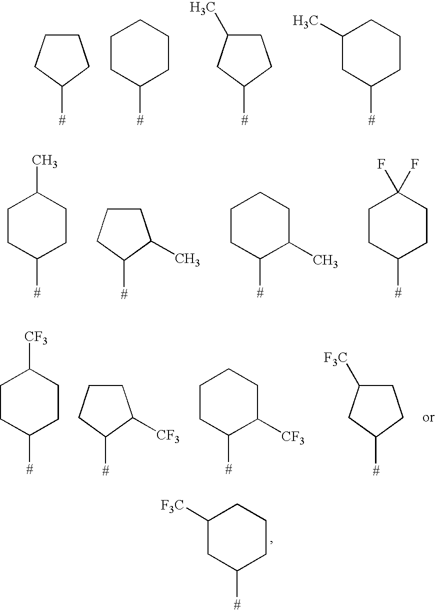
   where # in each case marks the point of attachment.

Very particular preference is likewise given to compounds of the formula I in which
R¹ represents a radical of the formula 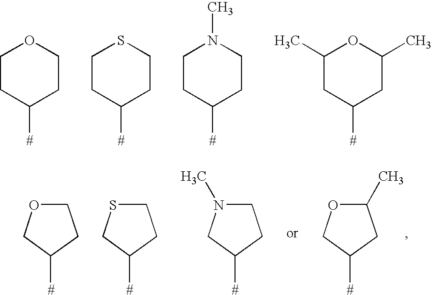
   where # in each case marks the point of attachment.

Preference is given to compounds of the formula (I) in which R² is an aromatic heterocycle.

Moreover, preference is given to compounds of the formula (I) in which R² is a three-, five- or six-membered, and in particular a five-membered, heterocycle.

Especially preferred are compounds of the formula (I) in which R² is a nitrogen-containing heterocycle.

In addition, preference is given to compounds of the formula (I) in which R² is a heterocycle which is attached via nitrogen to the pyrimidine ring.

Preference is likewise given to compounds of the formula (I) in which R² represents the following groups: pyrrole, pyrazole, imidazole, 1,2,4-triazole, 1,2,3-triazole, tetrazole, 1,2,3-triazine, 1,2,4-triazine, oxazole, isoxazole, 1,3,4-oxadiazole, 1,3,4-thiadiazole, furan, thiophene, thiazole, isothiazole, where the heterocycle is attached via carbon or nitrogen to the pyrimidine ring.

Preference is furthermore given to compounds of the formula (I) in which the cycle R² represents pyridazine, pyrimidine or pyrazine.

Preference is likewise given to compounds of the formula (I) in which R² represents pyrazole, pyrrole, imidazole, 1,2,3-triazole, 1,2,4-triazole, 1,3,4-oxadiazole, 1,3,4-thiadiazole, tetrazole, 2-pyridine, 2-pyrimidine, pyrazine or 3-pyridazine, optionally substituted by up to three groups R^{b}.

Particular preference is given to compounds of the formula (I) in which R² represents pyrazole, 1,2,3-triazole, 1,2,4-triazole or pyridazine.

In addition, particular preference is given to compounds of the formula (I) in which the cycle R² is substituted by one to three identical or different of the groups R^{b} below:
halogen, hydroxyl, cyano, nitro, amino, mercapto, C₁-C₄-alkyl, C₁-C₄-haloalkyl, C₂-C₄-alkenyl, C₂-C₆-alkynyl, C₃-C₆-cycloalkyl, C₁-C₄-alkoxy, C₁-C₄-haloalkoxy, carboxyl, C₁-C₄-alkoxycarbonyl, carbamoyl, C₁-C₇-alkylaminocarbonyl, C₁-C₆-alkyl-C₁-C₆-alkylaminocarbonyl, morpholinocarbonyl, pyrrolidinocarbonyl, C₁-C₄-alkylcarbonylamino, C₁-C₄-alkylamino, di-(C₁-C₄-alkyl)amino, C₁-C₄-alkylthio, C₁-C₄-alkylsulphinyl, C₁-C₄-alkylsulphonyl, hydroxysulphonyl, aminosulphonyl, C₁-C₄-alkylaminosulphonyl or di-(C₁-C₄-alkyl)aminosulphonyl.

Particular preference is in particular given to compounds of the formula (I) in which the cycle R² is substituted by one to three identical or different of the groups R^{b} below:
halogen, cyano, nitro, amino, C₁-C₄-alkyl, C₁-C₄-haloalkyl, C₁-C₄-alkoxy, carboxyl, C₁-C₄-alkoxycarbonyl, carbamoyl, C₁-C₄-alkylaminocarbonyl, di-(C₁-C₆-alkyl)aminocarbonyl or C₁-C₄-alkylcarbonylamino.

Particular preference is given to compounds of the formula (I) in which R² is unsubstituted or monosubstituted by halogen, cyanol, nitro, methyl or methoxy.

Moreover, particular preference is given to compounds of the formula (I) in which R³ represents halogen, C₁-C₆-alkyl, C₁-C₆-haloalkyl or C₁-C₆-alkoxy, in particular halogen.

Particular preference is given to compounds of the formula (I) in which R³ represents chlorine.

Preference is furthermore given to compounds of the formula (I) in which R⁴ and R⁸ do not represent hydrogen.

Moreover, preference is given to compounds of the formula (I) in which R⁴ represents hydrogen.

Particular preference is given to compounds of the formula (I) in which R⁴ represents hydrogen or halogen and R⁸ represents halogen or methyl.

In addition, particular preference is given to compounds of the formula (I) in which R⁵, R⁶ and R⁷ are unsubstituted.

Furthermore, particular preference is given to compounds I in which R⁵ and R⁷ are identical or different and represent hydrogen or halogen.

Moreover, particular preference is given to compounds I in which R⁶ represents hydrogen, halogen, C₁-C₄-alkyl, C₁-C₄-alkoxy, C₁-C₄-haloalkoxy, C₁-C₄-alkoxycarbonyl, C₁-C₄-haloalkoxycarbonyl, aminocarbonyl, alkylaminocarbonyl, dialkylaminocarbonyl, C₁-C₄-alkylcarbonyl, C₁-C₄-alkylmercapto, C₁-C₄-alkylsulphinyl, C₁-C₄-alkylsulphonyl, alkylaminosulphonyl or dialkylaminosulphonyl.

In particular with a view to their use, preference is given to the compounds I compiled in the tables below. The groups mentioned a the substituent in the table are furthermore, independently of the combination in which they are mentioned, a particularly preferred embodiment of the substituent in question.

Table 1

Compounds of the formula I-1, in which R⁸ represents fluorine, R⁴ represents chlorine and R⁵, R⁶ and R⁷ represent hydrogen and R¹ for a compound corresponds in each case to one of the radicals specified in Table A. 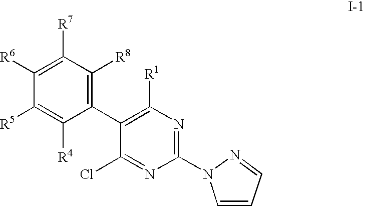
Table 2

Compounds of the formula I-1, in which R⁸ and R⁴ represent fluorine and R⁵, R⁶ and R⁷ represent hydrogen and R¹ for a compound corresponds in each case to one of the radicals specified in Table A.

Table 3

Compounds of the formula I-1, in which R⁸ and R⁴ represent chlorine and R⁵, R⁶ and R⁷ represent hydrogen and R¹ for a compound corresponds in each case to one of the radicals specified in Table A.

Table 4

Compounds of the formula I-1, in which R⁸ represents fluorine and R⁴ represents methyl and R⁵, R⁶ and R⁷ represent hydrogen and R¹ for a compound corresponds in each case to one of the radicals specified in Table A.

Table 5

Compounds of the formula I-1, in which R⁸, R⁴ and R⁶ represent fluorine and R⁵ and R⁷ represent hydrogen and R¹ for a compound corresponds in each case to one of the radicals specified in Table A.

Table 6

Compounds of the formula I-1, in which R⁸ and R⁴ represent fluorine, R⁵ and R⁷ represent hydrogen and R⁶ represents methoxy and R¹ for a compound corresponds in each case to one of the radicals specified in Table A.

Table 7

Compounds of the formula I-1, in which R⁸, R⁴, R⁵, R⁶ and R⁷ represent fluorine and R¹ for a compound corresponds in each case to one of the radicals specified in Table A.

Table 8

Compounds of the formula I-1, in which R⁸ represents methyl, R⁴, R⁵ and R⁷ represent hydrogen and R⁶ represents fluorine and R¹ for a compound corresponds in each case to one of the radicals specified in Table A.

Table 9

Compounds of the formula I-1, in which R⁸ and R⁶ represent fluorine, R⁴, R⁵ and R⁷ represent hydrogen and R¹ for a compound corresponds in each case to one of the radicals specified in Table A.

Table 10

Compounds of the formula I-1, in which R⁸ and R⁶ represent methyl and R⁴, R⁵ and R⁷ represent hydrogen and R¹ for a compound corresponds in each case to one of the radicals specified in Table A.

Table 11

Compounds of the formula I-1, in which R⁸ represents fluorine, R⁶ represents methyl and R⁴, R⁵ and R⁷ represent hydrogen and R¹ for a compound corresponds in each case to one of the radicals specified in Table A.

Table 12

Compounds of the formula I-1, in which R⁸ represent chlorine, R⁴ and R⁶ represent fluorine and R⁵ and R⁷ represent hydrogen and R¹ for a compound corresponds in each case to one of the radicals specified in Table A.

Table 13

Compounds of the formula I-1, in which R⁸ represents chlorine, R⁶ represents fluorine and R⁴, R⁵ and R⁷ represent hydrogen and R¹ for a compound corresponds in each case to one of the radicals specified in Table A.

Table 14

Compounds of the formula I-1, in which R⁸ and R⁶ represent chlorine and R⁴, R⁵ and R⁷ represent hydrogen and R¹ for a compound corresponds in each case to one of the radicals specified in Table A.

Table 15

Compounds of the formula I-1, in which R⁸ represents methyl, R⁴ and R⁶ represent fluorine and R⁵ and R⁷ represent hydrogen and R¹ for a compound corresponds in each case to one of the radicals specified in Table A.

Table 16

Compounds of the formula I-2, in which R⁸ represents fluorine, R⁴ represents chlorine and R⁵, R⁶ and R⁷ represent hydrogen and R¹ for a compound corresponds in each case to one of the radicals specified in Table A. 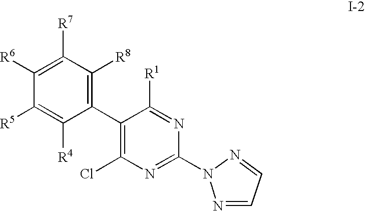
Table 17

Compounds of the formula I-2, in which R⁸ and R⁴ represent fluorine and R⁵, R⁶ and R⁷ represent hydrogen and R¹ for a compound corresponds in each case to one of the radicals specified in Table A.

Table 18

Compounds of the formula I-2, in which R⁸ and R⁴ represent chlorine and R⁵, R⁶ and R⁷ represent hydrogen and R¹ for a compound corresponds in each case to one of the radicals specified in Table A.

Table 19

Compounds of the formula I-2, in which R⁸ represents fluorine and R⁴ represents methyl and R⁵, R⁶ and R⁷ represent hydrogen and R¹ for a compound corresponds in each case to one of the radicals specified in Table A.

Table 20

Compounds of the formula I-2, in which R⁸, R⁴ and R⁶ represent fluorine and R⁵ and R⁷ represent hydrogen and R¹ for a compound corresponds in each case to one of the radicals specified in Table A.

Table 21

Compounds of the formula I-2, in which R⁸ and R⁴ represent fluorine, R⁵ and R⁷ represent hydrogen and R⁶ represents methoxy and R¹ for a compound corresponds in each case to one of the radicals specified in Table A.

Table 22

Compounds of the formula I-2, in which R⁸, R⁴, R⁵, R⁶ and R⁷ represent fluorine and R¹ for a compound corresponds in each case to one of the radicals specified in Table A.

Table 23

Compounds of the formula I-2, in which R⁸ represents methyl, R⁴, R⁵ and R⁷ represent hydrogen and R⁶ represents fluorine and R¹ for a compound corresponds in each case to one of the radicals specified in Table A.

Table 24

Compounds of the formula I-2, in which R⁸ and R⁶ represent fluorine, R⁴, R⁵ and R⁷ represent hydrogen and R¹ for a compound corresponds in each case to one of the radicals specified in Table A.

Table 25

Compounds of the formula I-2, in which R⁸ and R³ represent methyl and R⁴, R⁵ and R⁷ represent hydrogen and R¹ for a compound corresponds in each case to one of the radicals specified in Table A.

Table 26

Compounds of the formula I-2, in which R⁸ represents fluorine, R⁶ represents methyl and R⁴, R⁵ and R⁷ represent hydrogen and R¹ for a compound corresponds in each case to one of the radicals specified in Table A.

Table 27

Compounds of the formula I-2, in which R⁸ represents chlorine, R⁴ and R⁶ represent fluorine and R⁵ and R⁷ represent hydrogen and R¹ for a compound corresponds in each case to one of the radicals specified in Table A.

Table 28

Compounds of the formula I-2, in which R⁸ represents chlorine, R⁶ represents fluorine and R⁴, R⁵ and R⁷ represent hydrogen and R¹ for a compound corresponds in each case to one of the radicals specified in Table A.

Table 29

Compounds of the formula I-2, in which R⁸ and R⁶ represent chlorine and R⁴, R⁵ and R⁷ represent hydrogen and R¹ for a compound corresponds in each case to one of the radicals specified in Table A.

Table 30

Compounds of the formula I-2, in which R⁸ represents methyl, R⁴ and R⁶ represent fluorine and R⁵ and R⁷ represent hydrogen and R¹ for a compound corresponds in each case to one of the radicals specified in Table A.

Table 31

Compounds of the formula I-3, in which R⁸ represents fluorine, R⁴ represents chlorine and R⁵, R⁶ and R⁷ represent hydrogen and R¹ for a compound corresponds in each case to one of the radicals specified in Table A. 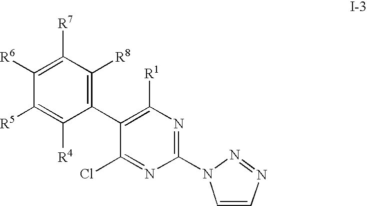
Table 32

Compounds of the formula I-3, in which R⁸ and R⁴ represent fluorine and R⁵, R⁶ and R⁷ represent hydrogen and R¹ for a compound corresponds in each case to one of the radicals specified in Table A.

Table 33

Compounds of the formula I-3, in which R⁸ and R⁴ represent chlorine and R⁵, R⁶ and R⁷ represent hydrogen and R¹ for a compound corresponds in each case to one of the radicals specified in Table A.

Table 34

Compounds of the formula I-3, in which R⁸ represents fluorine and R⁴ represents methyl and R⁵, R⁶ and R⁷ represent hydrogen and R¹ for a compound corresponds in each case to one of the radicals specified in Table A.

Table 35

Compounds of the formula I-3, in which R⁸, R⁴ and R⁶ represent fluorine and R⁵ and R⁷ represent hydrogen and R¹ for a compound corresponds in each case to one of the radicals specified in Table A.

Table 36

Compounds of the formula I-3, in which R⁸ and R⁴ represent fluorine, R⁵ and R⁷ represent hydrogen and R⁶ represents methoxy and R¹ for a compound corresponds in each case to one of the radicals specified in Table A.

Table 37

Compounds of the formula I-3, in which R⁸, R⁴, R⁵, R⁶ and R⁷ represent fluorine and R¹ for a compound corresponds in each case to one of the radicals specified in Table A.

Table 38

Compounds of the formula I-3, in which R⁸ represents methyl, R⁴, R⁵ and R⁷ represent hydrogen and R⁶ represents fluorine and R¹ for a compound corresponds in each case to one of the radicals specified in Table A.

Table 39

Compounds of the formula I-3, in which R⁸ and R⁶ represent fluorine, R⁴, R⁵ and R⁷ represent hydrogen and R¹ for a compound corresponds in each case to one of the radicals specified in Table A.

Table 40

Compounds of the formula I-3, in which R⁸ and R⁶ represent methyl and R⁴, R⁵ and R⁷ represent hydrogen and R¹ for a compound corresponds in each case to one of the radicals specified in Table A.

Table 41

Compounds of the formula I-3, in which R⁸ represents fluorine, R⁶ represents methyl and R⁴, R⁵ and R⁷ represent hydrogen and R¹ for a compound corresponds in each case to one of the radicals specified in Table A.

Table 42

Compounds of the formula I-3, in which R⁸ represents chlorine, R⁴ and R⁶ represent fluorine and R⁵ and R⁷ represent hydrogen and R¹ for a compound corresponds in each case to one of the radicals specified in Table A.

Table 43

Compounds of the formula I-3, in which R⁸ represents chlorine, R⁶ represents fluorine and R⁴, R⁵ and R⁷ represent hydrogen and R¹ for a compound corresponds in each case to one of the radicals specified in Table A.

Table 44

Compounds of the formula I-3, in which R⁸ and R⁶ represent chlorine and R⁴, R⁵ and R⁷ represent hydrogen and R¹ for a compound corresponds in each case to one of the radicals specified in Table A.

Table 45

Compounds of the formula I-3, in which R⁸ represents methyl, R⁴ and R⁶ represent fluorine and R⁵ and R⁷ represent hydrogen and R¹ for a compound corresponds in each case to one of the radicals specified in Table A.

Table 46

Compounds of the formula I-4, in which R⁸ represents fluorine, R⁴ represents chlorine and R⁵, R⁶ and R⁷ represent hydrogen and R¹ for a compound corresponds in each case to one of the radicals specified in Table A. 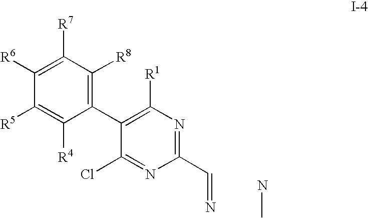
Table 47

Compounds of the formula I-4, in which R⁸ and R⁴ represent fluorine and R⁵, R⁶ and R⁷ represent hydrogen and R¹ for a compound corresponds in each case to one of the radicals specified in Table A.

Table 48

Compounds of the formula I-4, in which R⁸ and R⁴ represent chlorine and R⁵, R⁶ and R⁷ represent hydrogen and R¹ for a compound corresponds in each case to one of the radicals specified in Table A.

Table 49

Compounds of the formula I-4, in which R⁸ represents fluorine and R⁴ represents methyl and R⁵, R⁶ and R⁷ represent hydrogen and R¹ for a compound corresponds in each case to one of the radicals specified in Table A.

Table 50

Compounds of the formula I-4, in which R⁸, R⁴ and R⁶ represent fluorine and R⁵ and R⁷ represent hydrogen and R¹ for a compound corresponds in each case to one of the radicals specified in Table A.

Table 51

Compounds of the formula I-4, in which R⁸ and R⁴ represent fluorine, R⁵ and R⁷ represent hydrogen and R⁶ represents methoxy and R¹ for a compound corresponds in each case to one of the radicals specified in Table A.

Table 52

Compounds of the formula I-4, in which R⁸, R⁴, R⁵, R⁶ and R⁷ represent fluorine and R¹ for a compound corresponds in each case to one of the radicals specified in Table A.

Table 53

Compounds of the formula I-4, in which R⁸ represents methyl, R⁴, R⁵ and R⁷ represent hydrogen and R⁶ represents fluorine and R¹ for a compound corresponds in each case to one of the radicals specified in Table A.

Table 54

Compounds of the formula I-4, in which R⁸ and R⁶ represent fluorine, R⁴, R⁵ and R⁷ represent hydrogen and R¹ for a compound corresponds in each case to one of the radicals specified in Table A.

Table 55

Compounds of the formula I-4, in which R⁸ and R⁶ represent methyl and R⁴, R⁵ and R⁷ represent hydrogen and R¹ for a compound corresponds in each case to one of the radicals specified in Table A.

Table 56

Compounds of the formula I-4, in which R⁸ represents fluorine, R⁶ represents methyl and R⁴, R⁵ and R⁷ represent hydrogen and R¹ for a compound corresponds in each case to one of the radicals specified in Table A.

Table 57

Compounds of the formula I-4, in which R⁸ represents chlorine, R⁴ and R⁶ represent fluorine and R⁵ and R⁷ represent hydrogen and R¹ for a compound corresponds in each case to one of the radicals specified in Table A.

Table 58

Compounds of the formula I-4, in which R⁸ represents chlorine, R⁶ represents fluorine and R⁴, R⁵ and R⁷ represent hydrogen and R¹ for a compound corresponds in each case to one of the radicals specified in Table A.

Table 59

Compounds of the formula I-4, in which R⁸ and R⁶ represent chlorine and R⁴, R⁵ and R⁷ represent hydrogen and R¹ for a compound corresponds in each case to one of the radicals specified in Table A.

Table 60

Compounds of the formula I-4, in which R⁸ represents methyl, R⁴ and R⁶ represent fluorine and R⁵ and R⁷ represent hydrogen and R¹ for a compound corresponds in each case to one of the radicals specified in Table A.

Table 61

Compounds of the formula I-5, in which R⁸ represents fluorine, R⁴ represents chlorine and R⁵, R⁶ and R⁷ represent hydrogen and R¹ for a compound corresponds in each case to one of the radicals specified in Table A. 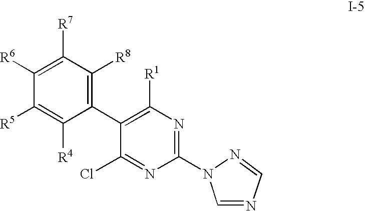
Table 62

Compounds of the formula I-5, in which R⁸ and R⁴ represent fluorine and R⁵, R⁶ and R⁷ represent hydrogen and R¹ for a compound corresponds in each case to one of the radicals specified in Table A.

Table 63

Compounds of the formula I-5, in which R⁸ and R⁴ represent chlorine and R⁵, R⁶ and R⁷ represent hydrogen and R¹ for a compound corresponds in each case to one of the radicals specified in Table A.

Table 64

Compounds of the formula I-5, in which R⁸ represents fluorine and R⁴ represents methyl and R⁵, R⁶ and R⁷ represent hydrogen and R¹ for a compound corresponds in each case to one of the radicals specified in Table A.

Table 65

Compounds of the formula I-5, in which R⁸, R⁴ and R⁶ represent fluorine and R⁵ and R⁷ represent hydrogen and R¹ for a compound corresponds in each case to one of the radicals specified in Table A.

Table 66

Compounds of the formula I-5, in which R⁸ and R⁴ represent fluorine, R⁵ and R⁷ represent hydrogen and R⁶ represents methoxy and R¹ for a compound corresponds in each case to one of the radicals specified in Table A.

Table 67

Compounds of the formula I-5, in which R⁸, R⁴, R⁵, R⁶ and R⁷ represent fluorine and R¹ for a compound corresponds in each case to one of the radicals specified in Table A.

Table 68

Compounds of the formula I-5, in which R⁸ represents methyl, R⁴, R⁵ and R⁷ represent hydrogen and R⁶ represents fluorine and R¹ for a compound corresponds in each case to one of the radicals specified in Table A.

Table 69

Compounds of the formula I-5, in which R⁸ and R⁶ represent fluorine, R⁴, R⁵ and R⁷ represent hydrogen and R¹ for a compound corresponds in each case to one of the radicals specified in Table A.

Table 70

Compounds of the formula I-5, in which R⁸ and R⁶ represent methyl and R⁴, R⁵ and R⁷ represent hydrogen and R¹ for a compound corresponds in each case to one of the radicals specified in Table A.

Table 71

Compounds of the formula I-5, in which R⁸ represents fluorine, R⁶ represents methyl and R⁴, R⁵ and R⁷ represent hydrogen and R¹ for a compound corresponds in each case to one of the radicals specified in Table A.

Table 72

Compounds of the formula I-5, in which R⁸ represents chlorine, R⁴ and R⁶ represent fluorine and R⁵ and R⁷ represent hydrogen and R¹ for a compound corresponds in each case to one of the radicals specified in Table A.

Table 73

Compounds of the formula I-5, in which R⁸ represents chlorine, R⁶ represents fluorine and R⁴, R⁵ and R⁷ represent hydrogen and R¹ for a compound corresponds in each case to one of the radicals specified in Table A.

Table 74

Compounds of the formula I-5, in which R⁸ and R⁶ represent chlorine and R⁴, R⁵ and R⁷ represent hydrogen and R¹ for a compound corresponds in each case to one of the radicals specified in Table A.

Table 75

Compounds of the formula I-5, in which R⁸ represents methyl, R⁴ and R⁶ represent fluorine and R⁵ and R⁷ represent hydrogen and R¹ for a compound corresponds in each case to one of the radicals specified in Table A.

TABLE A
# in each case marks the point of attachment.

| No. | R¹ |
|---|---|
| A-1 | 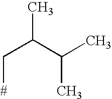 |
| A-2 | 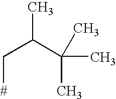 |
| A-3 | 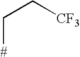 |
| A-4 | 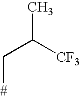 |
| A-5 | 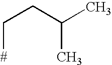 |
| A-6 | 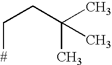 |
| A-7 | 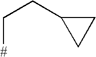 |
| A-8 | 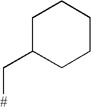 |
| A-9 | 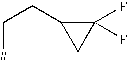 |
| A-10 | 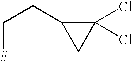 |
| A-11 | 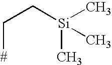 |
| A-12 | 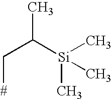 |
| A-13 | 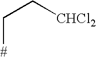 |
| A-14 | 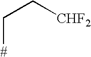 |
| A-15 | 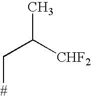 |
| A-16 | 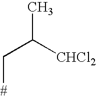 |
| A-17 | 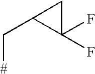 |
| A-18 | 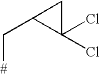 |
| A-19 | 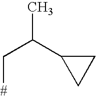 |
| A-20 | 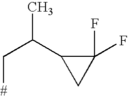 |
| A-21 | 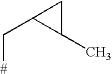 |
| A-22 | 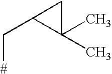 |
| A-23 | 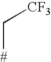 |
| A-24 | 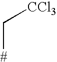 |
| A-25 | 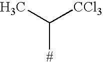 |
| A-26 | 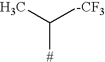 |
| A-27 | 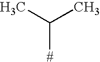 |
| A-28 | 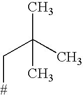 |
| A-29 | 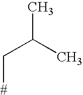 |
| A-30 | 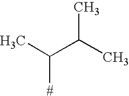 |
| A-31 | 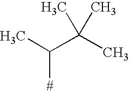 |
| A-32 | 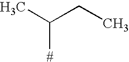 |
| A-33 | 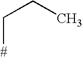 |
| A-34 | 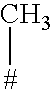 |
| A-35 | 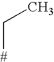 |
| A-36 | 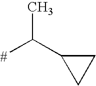 |
| A-37 | 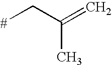 |
| A-38 | 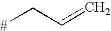 |
| A-39 | 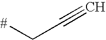 |
| A-40 | 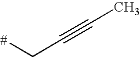 |
| A-41 | 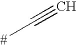 |
| A-42 | 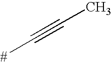 |
| A-43 | 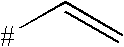 |
| A-44 | 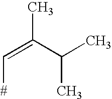 |
| A-45 | 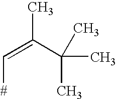 |
| A-46 | 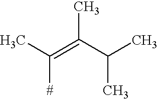 |
| A-47 | 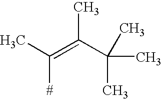 |
| A-48 | 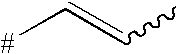 |
| A-49 | 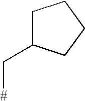 |
| A-50 | 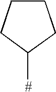 |
| A-51 | 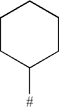 |
| A-52 | 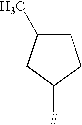 |
| A-53 | 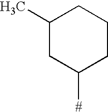 |
| A-54 | 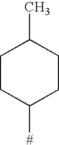 |
| A-55 | 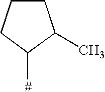 |
| A-56 | 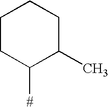 |
| A-57 | 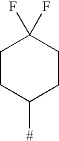 |
| A-58 | 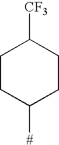 |
| A-59 | 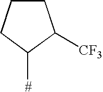 |
| A-60 | 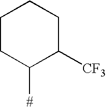 |
| A-61 | 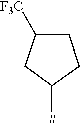 |
| A-62 | 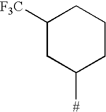 |
| A-63 | 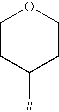 |
| A-64 | 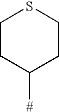 |
| A-65 | 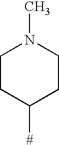 |
| A-66 | 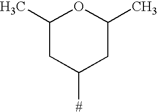 |
| A-67 | 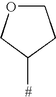 |
| A-68 | 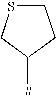 |
| A-69 | 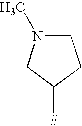 |
| A-70 | 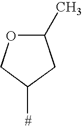 |

Suitable diluents for carrying out the process a) according to the invention are acids, such as acetic acid, formic acid, alcohols, such as methanol, water or halogenated hydrocarbons, such as dichloromethane or chloroform. It is also possible to use mixtures of these solvents. Preference is given to acetic acid or, in the case where the oxidizing agent is Oxone, methanol/water mixtures.

Suitable oxidizing agents for carrying out the process a) according to the invention are, for example, hydrogen peroxide, pertungstic acid, peracetic acid, 3-chlorobenzoic acid, perphthalic acid, chlorine, oxygen and Oxone® (KHSO₅).

Suitable acidic receptors for carrying out the process a) according to the invention are all inorganic and organic bases customary for such reactions. Preference is given to using alkaline earth metal or alkali metal hydroxides, acetates, carbonates, bicarbonates or phosphates, such as, for example, sodium hydroxide, potassium hydroxide, sodium acetate, sodium carbonate, potassium carbonate, potassium bicarbonate, sodium bicarbonate, caesium carbonate or silver phosphate.

When carrying out the process a) according to the invention, the reaction temperatures can be varied within a relatively wide range. In general, the process is carried out at temperatures of from 0° C. to 100° C., preferably at temperatures of from 10° C. to 50° C. (cf. WO 02/074753 and the literature cited therein).

For carrying out the process a) according to the invention for preparing the compounds of the formula (I), in general from 1 to 5 mol, preferably from 1 to 2 mol, of the compound of the formula (IV) are employed per mole of the compound of the formula (II).

The formula (II) provides a general definition of the compounds required as starting materials for carrying out the process a) according to the invention. In this formula, R¹, R⁴, R⁵, R⁶, R⁷, R⁸ and Hal preferably and in particular have those meanings which have already been mentioned in connection with the description of the compounds of the formula (I) according to the invention as being preferred.

The starting materials of the formula (II) are novel and also form part of the subject-matter of the invention.

The formula (IV) defines the starting materials also required for carrying out the process a) according to the invention. In this formula, R² preferably and in particular has those meanings which have already been mentioned in connection with the description of the compounds of the formula (I) according to the invention as being preferred.

The starting materials of the formula (IV) are known and/or can be prepared by known methods.

The starting materials of the formula (II) are obtained when (process e)) compounds of the formula 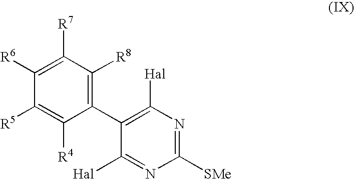
in which
R⁴, R⁵, R⁶, R⁷, R⁸ and Hal are as defined above are reacted with a compound of the formula
   R¹-M¹ (VI),
in which
R¹ is as defined above and
M¹ represents lithium, sodium, potassium, dihydroxyboranyl, a radical of the formula 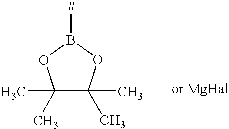
   in which
   Hal represents chlorine, bromine or iodine,
      if appropriate in the presence of a diluent and if appropriate in the presence of a catalyst.

The metal compounds of the formula (VI) are known or can be prepared by known methods.

The compounds of the formula (XI) are likewise known (cf. WO 02/074753) or they can be prepared by known methods.

Suitable diluents for carrying out the process e) according to the invention are all inert organic solvents which are customary for such reactions. Preference is given to using ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; amides, such as N,N-dimethylformamide, N,N-dimethylacetamide, N-methylformanilide, N-methylpyrrolidone or hexamethylphosphoric triamide.

Suitable catalysts for carrying out the process e) according to the invention are all reaction promoters customary for such reactions. Preference is given to using palladium, nickel, copper or iron salts or complexes. Copper(I) chloride, copper(I) bromide, copper(I) iodide, copper(I) cyanide, iron(III) acetate, iron(III) acetylacetonate, tetrakis(triphenylphosphine)palladium, bis(triphenylphosphine)palladium dichloride and 1,1′-bis(diphenylphosphino)ferrocenepalladium(II) chloride may be mentioned by way of example.

Preference is also given to using palladium or nickel complexes generated in the reaction mixture by separately adding a palladium or nickel salt and a substance acting as complex ligand to the reaction mixture. Examples of ligand formers which may be mentioned are:
triethylphosphine, tri-tert-butylphosphine, tricyclohexylphosphine, 2-(dicyclohexylphosphine)biphenyl, 2-(di-tert-butylphosphine)biphenyl, 2-(dicyclohexylphosphine)-2′-(N,N-dimethylamino)-biphenyl, triphenylphosphine, tris-(o-tolyl)-phosphine, sodium 3-(diphenylphosphino)benzenesulphonate, tris-2-(methoxyphenyl)phosphine, 2,2′-bis-(diphenylphosphine)-1,1′-binaphthyl, 1,4-bis-(diphenylphosphine)butane, 1,2-bis-(diphenylphosphine)ethane, 1,4-bis-(dicyclohexylphosphine)butane, 1,2-bis-(dicyclohexylphosphine)ethane, 2-(dicyclohexylphosphine)-2′-(N,N-dimethylamino)biphenyl, bis(diphenylphosphino)ferrocene and tris-(2,4-tert-butylphenyl)-phosphite.

When carrying out the first step of the process e) according to the invention, the reaction temperatures can be varied within a relatively wide range. In general, the first step of the process is carried out at temperatures between 0° C. and 150° C., preferably at temperatures between 0° C. and 80° C.

When carrying out the first step of the process e) according to the invention, in general from 1 to 10 mol, preferably from 1 to 3 mol, of a metal compound of the formula (VI) are employed per mole of the compound of the formula (XI). Work-up is carried out by customary methods.

Suitable diluents for carrying out the process b) according to the invention are all inert organic solvents which are customary for such reactions. Preference is given to using ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; amides, such as N,N-dimethylformamide, N,N-dimethylacetamide, N-methylformanilide, N-methylpyrrolidone or hexamethylphosphoric triamide.

Suitable catalysts for carrying out the process b) according to the invention are all reaction promoters customary for such reactions. Preference is given to using palladium, nickel, copper or iron salts or complexes. Copper(I) chloride, copper(I) bromide, copper(I) iodide, copper(I) cyanide, iron(III) acetate, iron(III) acetylacetonate, tetrakis(triphenylphosphine)palladium, bis(triphenylphosphine) palladium dichloride and 1,1′-bis(diphenylphosphino)ferrocenepalladium(II) chloride may be mentioned by way of example.

Preference is also given to using palladium or nickel complexes generated in the reaction mixture by separately adding a palladium or nickel salt and a substance acting as complex ligand to the reaction mixture. Examples of ligand formers which may be mentioned are:
triethylphosphine, tri-tert-butylphosphine, tricyclohexylphosphine, 2-(dicyclohexylphosphine)biphenyl, 2-(di-tert-butylphosphine)biphenyl, 2-(dicyclohexylphosphine)-2′-(N,N-dimethylamino)-biphenyl, triphenylphosphine, tris-(o-tolyl)-phosphine, sodium 3-(diphenylphosphino)benzenesulphonate, tris-2-(methoxyphenyl)phosphine, 2,2′-bis-(diphenylphosphine)-1,1′-binaphthyl, 1,4-bis-(diphenylphosphine)butane, 1,2-bis-(diphenylphosphine)ethane, 1,4-bis-(dicyclohexylphosphine)butane, 1,2-bis-(dicyclohexylphosphine)ethane, 2-(dicyclohexylphosphine)-2′-(N,N-dimethylamino)biphenyl, bis(diphenylphosphino)ferrocene and tris-(2,4-tert-butylphenyl)-phosphite.

When carrying out the first step of the process b) according to the invention, the reaction temperatures can be varied within a relatively wide range. In general, the first step of the process is carried out at temperatures between 0° C. and 150° C., preferably at temperatures between 0° C. and 80° C.

When carrying out the first step of the process b) according to the invention, in general from 1 to 10 mol, preferably from 1 to 3 mol, of a metal compound of the formula (VI) are employed per mole of the compound of the formula (V). Work-up is carried out by customary methods.

The metal compounds of the formula (VI) are known or can be prepared by known methods.

The compounds of the formula (V) are likewise known (cf. WO 02/074753), or they can be prepared by known methods.

Suitable diluents for carrying out the process c1) according to the invention are all customary organic solvents. Preference is given to using ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; nitrites, such as acetonitrile, propionitrile, n- or i-butyronitrile or benzonitrile; amides, such as N,N-dimethylformamide, N,N-dimethylacetamide, N-methylformanilide, N-methylpyrrolidone or hexamethylphosphoric triamide; sulphoxides, such as dimethyl sulphoxide; sulphones, such as sulpholane; alcohols, such as methanol, ethanol, isopropanol, tert-butyl alcohol.

When carrying out the process c1) according to the invention, the reaction temperatures can be varied within a relatively wide range. In general, the process is carried out at temperatures of from 0° C. to 150° C., preferably at temperatures of from 20° C. to 100° C.

For carrying out the process c1) according to the invention for preparing the compounds of the formula (I′″), in general from 1 to 10 mol, preferably from 1 to 3 mol of the compound of the formula (VII) are generally employed per mole of the compound of the formula (I′) or (I″).

Suitable diluents for carrying out the process c2) according to the invention are all solvents customary for Grignard reactions. Preference is given to using ethers, such as diethyl ether or else tetrahydrofuran.

Suitable catalysts for carrying out the process c2) according to the invention are all reaction promoters mentioned for the process b) according to the invention.

When carrying out the process c2) according to the invention, the reaction temperatures can be varied within a certain range. In general, the process is carried out at temperatures between −20° C. and 80° C., preferably between 0° C. and 60° C.

For carrying out the process c2) according to the invention for preparing the compounds of the formula (I′″), in general from 1 to 10 mol, preferably from 1 to 3 mol, of the compound of the formula (VIII) are employed per mole of the compound of the formula (I′) or (I″).

The compounds of the formulae (I′) and I″) used as starting materials for carrying out the process c) according to the invention are obtained by the process a) or b) according to the invention. In the formulae (I′) and (I″), R¹, R², R⁴, R⁵, R⁶, R⁷, R⁸ and Hal preferably and in particular have those meanings which have already been given in connection with the description of the compounds of the formula (I) according to the invention as being preferred.

The formula (VII) provides a definition of the starting materials furthermore also required for carrying out the process c1) according to the invention. In this formula, R³ preferably and in particular has those meanings which have already been given in connection with the description of the compounds of the formula (I) according to the invention as being preferred. The compounds of the formula (VII) are known or can be prepared by known methods.

The formula (VIII) provides a definition of the starting materials furthermore also required for carrying out the process c2) according to the invention. In this formula, R³ preferably and in particular has those meanings which have already been given in connection with the description of the compounds of the formula (I) according to the invention as being preferred. The compounds of the formula (VIII) are known or can be prepared by known methods.

Suitable halogenating agents for carrying out the process d1) are all components customary for replacing hydroxyl groups by halogen. Preference is given to using phosphorus trichloride, phosphorus tribromide, phosphorus pentachloride, phosphorus oxychloride, thionyl chloride, thionyl bromide or mixtures thereof or phosgene, di- or triphosgene. If appropriate, chlorine is added to the halogenating agents mentioned or their mixtures. The corresponding fluoro compounds can be prepared from the chloro or bromo compounds by reaction with potassium fluoride.

Suitable diluents for carrying out the process d1) according to the invention are all solvents customary for such halogenations. Preference is given to using halogenated aliphatic or aromatic hydrocarbons, such as chlorobenzene. However, the halogenating agent itself, for example phosphorus oxychloride or a mixture of halogenating agents, may also act as diluent.

Suitable acid acceptors for carrying out the process d1) according to the invention are all inorganic and organic bases customary for such reactions. Preference is given to using alkaline earth metal or alkali metal hydroxides, acetates, carbonates, bicarbonates or phosphates, such as, for example, sodium hydroxide, potassium hydroxide, sodium acetate, sodium carbonate, potassium carbonate, potassium bicarbonate, sodium bicarbonate, caesium carbonate or silver phosphate.

When carrying out the process d1), the temperatures can also be varied within a relatively wide range. In general, the process is carried out at temperatures between 0° C. and 150° C., preferably between 10° C. and 120° C.

When carrying out the process d1), the compound of the formula (IX) is generally reacted with an excess of halogenating agents. Work-up is carried out by customary methods.

The formula (IX) provides a definition of the starting materials furthermore also required for carrying out the process d1) according to the invention. In this formula, R³, R⁴, R⁵, R⁶, R⁷, R⁸ and Hal preferably and in particular have those meanings which have already been given in connection with the description of the compounds of the formula (I) according to the invention as being preferred. The compounds of the formula (IX) are known (cf. WO 02/074753), or they can be prepared by known methods.

Suitable diluents for carrying out the process d2) according to the invention are all inert organic solvents which are customary for such reactions. Preference is given to using ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; amides, such as N,N-dimethylformamide, N,N-dimethylacetamide, N-methylformanilide, N-methylpyrrolidone or hexamethylphosphoric triamide.

Suitable catalysts for carrying out the process d2) according to the invention are all reaction promoters customary for such reactions. Preference is given to using palladium, nickel, copper or iron salts or complexes. Copper(I) chloride, copper(I) bromide, copper(I) iodide, copper(I) cyanide, iron(III) acetate, iron(III) acetylacetonate, tetrakis(triphenylphosphine)palladium, bis(triphenylphosphine)palladium dichloride and 1,1′-bis(diphenylphosphino)ferrocenepalladium(II) chloride may be mentioned by way of example.

Preference is also given to using palladium or nickel complexes generated in the reaction mixture by separately adding a palladium or nickel salt and a substance acting as complex ligand to the reaction mixture. Examples of ligand formers which may be mentioned are:
triethylphosphine, tri-tert-butylphosphine, tricyclohexylphosphine, 2-(dicyclohexylphosphine)biphenyl, 2-(di-tert-butylphosphine)biphenyl, 2-(dicyclohexylphosphine)-2′-(N,N-dimethylamino)-biphenyl, triphenylphosphine, tris-(o-tolyl)-phosphine, sodium 3-(diphenylphosphino)benzenesulphonate, tris-2-(methoxyphenyl)phosphine, 2,2′-bis-(diphenylphosphine)-1,1′-binaphthyl, 1,4-bis-(diphenylphosphine)butane, 1,2-bis-(diphenylphosphine)ethane, 1,4-bis-(dicyclohexylphosphine)butane, 1,2-bis-(dicyclohexylphosphine)ethane, 2-(dicyclohexylphosphine)-2′-(N,N-dimethylamino)biphenyl, bis(diphenylphosphino)ferrocene and tris-(2,4-tert-butylphenyl)-phosphite.

When carrying out the first step of the process d2) according to the invention, the reaction temperatures can be varied within a relatively wide range. In general, the first step of the process is carried out at temperatures between 0° C. and 150° C., preferably at temperatures between 0° C. and 80° C.

When carrying out the first step of the process d2) according to the invention, in general from 1 to 10 mol, preferably from 1 to 3 mol, of a metal compound of the formula (VI) are employed per mole of the compound of the formula (X). Work-up is carried out by customary methods.

The metal compounds of the formula (VI) are known or can be prepared by known methods.

The processes a), b), c), d) and e) according to the invention are generally carried out under atmospheric pressure. However, it is also possible to operate under elevated pressure.

The compounds according to the invention inhibit the growth of tumour cells and related diseases in mammals and can be used as medicaments. They are particularly suitable for preparing medicaments for controlling cancer.

The invention furthermore relates to a method for inhibiting the growth of cancer-like tumour cells and related diseases in a mammal requiring this treatment. This method comprises administering an effective amount of a 5-phenylpyrimidine or a pharmaceutically acceptable salt thereof to a mammal. The invention furthermore relates to a method for treating or hindering the growth of tumour cells and related diseases by interaction with tubulin and microtubuli and promoting the polymerization of microtubuli by administering an effective amount of a 5-phenylpyrimidine or a pharmaceutically effective salt thereof to a mammal.

The compounds according to the invention have potent microbicidal activity and can be employed for controlling unwanted microorganisms, such as fungi and bacteria, in crop protection and in the protection of materials.

Fungicides can be employed in crop protection for controlling Plasmodiophoromycetes, Oomycetes, Chytridiomycetes, Zygomycetes, Ascomycetes, Basidiomycetes and Deuteromycetes.

Bactericides can be employed in crop protection for controlling Pseudomonadaceae, Rhizobiaceae, Enterobacteriaceae, Corynebacteriaceae and Streptomycetaceae.

Some pathogens causing fungal and bacterial diseases which come under the generic names listed above may be mentioned as examples, but not by way of limitation:
Xanthomonias species, such as, for example, Xanthomonas campestris pv. oryzae;
Pseudomonas species, such as, for example, Pseudomonas syringae pv. lachrymans;
Erwinia species, such as, for example, Erwinia amylovora;
Pythium species, such as, for example, Pythium ultimum;
Phytophthora species, such as, for example, Phytophthora infestans;
Pseudoperonospora species, such as, for example, Pseudoperonospora humuli or Pseudoperonospora cubensis;
Plasmopara species, such as, for example, Plasmopara viticola;
Bremia species, such as, for example, Bremia lactucae;
Peronospora species, such as, for example, Peronospora pisi or P. brassicae;
Erysiphe species, such as, for example, Erysiphe graminis;
Sphaerotheca species, such as, for example, Sphaerotheca fuliginea;
Podosphaera species, such as, for example, Podosphaera leucotricha;
Venturia species, such as, for example, Venturia inaequalis;
Pyrenophora species, such as, for example, Pyrenophora teres or P. graminea (conidia form: Drechslera, syn: Helminthosporium);
Cochliobolus species, such as, for example, Cochliobolus sativus (conidia form: Drechslera, syn: Helminthosporium);
Uromyces species, such as, for example, Uromyces appendiculatus;
Puccinia species, such as, for example, Puccinia recondita;
Sclerotinia species, such as, for example, Sclerotinia sclerotiorum;
Tilletia species, such as, for example, Tilletia caries;
Ustilago species, such as, for example, Ustilago nuda or Ustilago avenae;
Pellicularia species, such as, for example, Pellicularia sasakii;
Pyricularia species, such as, for example, Pyricularia oryzae;
Fusarium species, such as, for example, Fusarium culmorum;
Botrytis species, such as, for example, Botrytis cinerea;
Septoria species, such as, for example, Septoria nodorum;
Leptosphaeria species, such as, for example, Leptosphaeria nodorum;
Cercospora species, such as, for example, Cercospora canescens;
Alternaria species, such as, for example, Alternaria brassicae; and
Pseudocercosporella species, such as, for example, Pseudocercosporella herpotrichoides.

The active compounds according to the invention also show a strong invigorating action in plants. Accordingly, they are suitable for mobilizing the internal defenses of the plant against attack by unwanted microorganisms.

In the present context, plant-invigorating (resistance-inducing) compounds are to be understood as meaning substances which are capable of stimulating the defense system of plants such that, when the treated plants are subsequently inoculated with unwanted microorganisms, they display substantial resistance to these microorganisms.

In the present case, unwanted microorganisms are to be understood as meaning phytopathogenic fungi, bacteria and viruses. The compounds according to the invention can thus be used to protect plants within a certain period of time after treatment against attack by the pathogens mentioned. The period of time for which this protection is achieved generally extends for 1 to 10 days, preferably 1 to 7 days, from the treatment of the plants with the active compounds.

The fact that the active compounds are well tolerated by plants at the concentrations required for controlling plant diseases permits the treatment of above-ground parts of plants, of propagation stock and seeds, and of the soil.

The active compounds according to the invention can be used with particularly good results for controlling cereal diseases, such as, for example, against Erysiphe species, of diseases in viticulture and in the cultivation of fruits and vegetables, such as, for example, against Botrytis, Venturia, Sphaerotheca and Podosphaera species.

The active compounds according to the invention are also suitable for increasing the yield of crops. In addition, they show reduced toxicity and are well tolerated by plants.

If appropriate, the active compounds according to the invention can, at certain concentrations and application rates, also be employed as herbicides, for regulating plant growth and for controlling animal pests. If appropriate, they can also be used as intermediates or precursors in the synthesis of other active compounds.

According to the invention, it is possible to treat all plants and parts of plants. Plants are to be understood here as meaning all plants and plant populations, such as desired and undesired wild plants or crop plants (including naturally occurring crop plants). Crop plants can be plants which can be obtained by conventional breeding and optimization methods or by biotechnological and genetic engineering methods or combinations of these methods, including the transgenic plants and including plant cultivars which can or cannot be protected by plant breeders' certificates. Parts of plants are to be understood as meaning all above-ground and below-ground parts and organs of plants, such as shoot, leaf, flower and root, examples which may be mentioned being leaves, needles, stems, trunks, flowers, fruit bodies, fruits and seeds and also roots, tubers and rhizomes. Parts of plants also include harvested material and vegetative and generative propagation material, for example seedlings, tubers, rhizomes, cuttings and seeds.

The treatment of the plants and parts of plants according to the invention with the active compounds is carried out directly or by action on their environment, habitat or storage area according to customary treatment methods, for example by dipping, spraying, evaporating, atomizing, broadcasting, brushing-on and, in the case of propagation material, in particular in the case of seeds, furthermore by one- or multilayer coating.

In the protection of materials, the compounds according to the invention can be employed for protecting industrial materials against infection with, and destruction by, unwanted microorganisms.

Industrial materials in the present context are understood as meaning non-living materials which have been prepared for use in industry. For example, industrial materials which are intended to be protected by active compounds according to the invention from microbial change or destruction can be tackifiers, sizes, paper and board, textiles, leather, wood, paints and plastic articles, cooling lubricants and other materials which can be infected with, or destroyed by, microorganisms. Parts of production plants, for example cooling-water circuits, which may be impaired by the proliferation of microorganisms may also be mentioned within the scope of the materials to be protected. Industrial materials which may be mentioned within the scope of the present invention are preferably tackifiers, sizes, papers and boards, leather, wood, paints, cooling lubricants and heat-transfer liquids, particularly preferably wood.

Microorganisms capable of degrading or changing the industrial materials which may be mentioned are, for example, bacteria, fungi, yeasts, algae and slime organisms. The active compounds according to the invention preferably act against fungi, in particular molds, wood-discoloring and wood-destroying fungi (Basidiomycetes) and against slime organisms and algae.

Microorganisms of the following genera may be mentioned as examples:
Alternaria, such as Alternaria tenuis,
Aspergillus, such as Aspergillus niger,
Chaetomium, such as Chaetomium globosum,
Coniophora, such as Coniophora puetana,
Lentinus, such as Lentinus tigrinus,
Penicillium, such as Penicillium glaucum,
Polyporus, such as Polyporus versicolor,
Aureobasidium, such as Aureobasidium pullulans,
Sclerophoma, such as Sclerophoma pityophila,
Trichoderma, such as Trichoderma viride,
Escherichia, such as Escherichia coli,
Pseudomonas, such as Pseudomonas aeruginosa, and
Staphylococcus, such as Staphylococcus aureus.

Depending on their particular physical and/or chemical properties, the active compounds can be converted into the customary formulations, such as solutions, emulsions, suspensions, powders, foams, pastes, granules, aerosols and microencapsulations in polymeric substances and in coating compositions for seeds, and ULV cool and warm fogging formulations.

These formulations are produced in a known manner, for example by mixing the active compounds with extenders, that is liquid solvents, liquefied gases under pressure, and/or solid carriers, optionally with the use of surfactants, that is emulsifiers and/or dispersants, and/or foam formers. If the extender used is water, it is also possible to employ, for example, organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and their ethers and esters, ketones such as acetone, methyl ethyl ketone, methyl isobutyl ketone or cyclohexanone, strongly polar solvents such as dimethylformamide or dimethyl sulphoxide, or else water. Liquefied gaseous extenders or carriers are to be understood as meaning liquids which are gaseous at standard temperature and under atmospheric pressure, for example aerosol propellants such as halogenated hydrocarbons, or else butane, propane, nitrogen and carbon dioxide. Suitable solid carriers are: for example ground natural minerals such as kaolins, clays, talc, chalk, quartz, attapulgite, montmorillonite or diatomaceous earth, and ground synthetic minerals such as finely divided silica, alumina and silicates. Suitable solid carriers for granules are: for example crushed and fractionated natural rocks such as calcite, pumice, marble, sepiolite and dolomite, or else synthetic granules of inorganic and organic meals, and granules of organic material such as sawdust, coconut shells, maize cobs and tobacco stalks. Suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates, or else protein hydrolysates. Suitable dispersants are: for example lignosulphite waste liquors and methylcellulose.

Tackifiers such as carboxymethylcellulose, natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic dyestuffs such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95 per cent by weight of active compound, preferably between 0.5 and 90%.

The active compounds according to the invention can, as such or in their formulations, also be used in a mixture with known fungicides, bactericides, acaricides, nematicides or insecticides, to broaden, for example, the activity spectrum or to prevent development of resistance. In many cases, synergistic effects are obtained, i.e. the activity of the mixture is greater than the activity of the individual components.

Suitable mixing components are, for example, the following compounds:

Fungicides:
2-phenylphenol; 8-hydroxyquinoline sulphate; acibenzolar-S-methyl; aldimorph; amidoflumet; ampropylfos; ampropylfos-potassium; andoprim; anilazine; azaconazole; azoxystrobin; benalaxyl; benodanil; benomyl; benthiavalicarb-isopropyl; benzamacril; benzamacril-isobutyl; bilanafos; binapacryl; biphenyl; bitertanol; blasticidin-S; bromuconazole; bupirimate; buthiobate; butylamine; calcium polysulphide; capsimycin; captafol; captan; carbendazim; carboxin; carpropamid; carvone; chinomethionat; chlobenthiazone; chlorfenazole; chloro-neb; chlorothalonil; chlozolinate; clozylacon; cyazofamid; cyflufenamid; cymo-xanil; cyproconazole; cyprodinil; cyprofuram; Dagger G; debacarb; dichlofluanid; dichlone; dichlorophen; diclocymet; diclomezine; dicloran; diethofencarb; difenoconazole; diflumetorim; dimethirimol; dimethomorph; dimoxystrobin; diniconazole; diniconazole-M; dinocap; diphenyl-amine; dipyrithione; ditalimfos; dithianon; dodine; drazoxolon; edifenphos; epoxiconazole; ethaboxam; ethirimol; etridiazole; famoxadone; fenamidone; fenapanil; fenarimol; fenbuconazole; fenfuram; fen-hexamid; fenitropaan; fenoxanil; fenpiclonil; fenpropidin; fenpropimorph; ferbam; fluazinam; flubenzimine; fludioxonil; flumetover; flumorph; fluoromide; fluoxastrobin; fluquinconazole; flurprimidol; flusilazole; flusulfamide; flutolanil; flutriafol; folpet; fosetyl-Al; fosetyl-sodium; fuberidazole; furalaxyl; furametpyr; furcarbanil; furmecyclox; guazatine; hexachlorobenzene; hexaconazole; hymexazole; imazalil; imibenconazole; iminoctadine triacetate; iminoctadine tris(albesil); iodocarb; ipconazole; iprobenfos; iprodione; iprovalicarb; irumamycin; isoprothiolane; isovaledione; kasugamycin; kresoxim-methyl; mancozeb; maneb; meferimzone; mepanipyrim; mepronil; metalaxyl; metalaxyl-M; metconazole; methasulfocarb; methfuroxam; metiram; metominostrobin; met-sulfovax; mildiomycin; myclobutanil; myclozolin; natamycin; nicobifen; nitrothal-isopropyl; noviflumuron; nuarimol; ofurace; orysastrobin; oxadixyl; oxolinic acid; oxpoconazole; oxycarboxin; oxyfenthiin; paclobutrazole; pefurazoate; penconazole; pencycuron; phosdiphen; phthalide; picoxystrobin; piperalin; polyoxins; polyoxorim; probenazole; prochloraz; procymidone; propamocarb; propanosine-sodium; propiconazole; propineb; proqiunazid; prothioconazole; pyraclostrobin; pyrazophos; pyrifenox; pyrimethanil; pyroquilon; pyroxyfur; pyrrolenitrine; quinconazole; quinoxyfen; quintozene; simeconazole; spiroxamine; sulphur; tebuconazole; tecloftalam; tecnazene; tetcyclacis; tetraconazole; thiabendazole; thicyofen; thifluzamide; thiophanate-methyl; thiram; tioxymid; tolclofos-methyl; tolylfluanid; triadimefon; triadimenol; triazbutil; triazoxide; tricyclamide; tri-cyclazole; tridemorph; trifloxystrobin; triflumizole; triforine; triticonazole; uniconazole; validamycin A; vinclozolin; zineb; ziram; zoxamide; (2S)-N-[2-[4-[[3-(4-chlorophenyl)-2-propynyl]oxy]-3-methoxyphenyl]ethyl]-3-methyl-2-[(methylsulfonyl)amino]butanamide; 1-(1-naphthalenyl)-1H-pyrrole-2,5-dione; 2,3,5,6-tetrachloro-4-(methylsulphonyl)pyridine; 2-amino-4-methyl-N-phenyl-5-thiazolecarboxamide; 2-chloro-N-(2,3-dihydro-1,1,3-trimethyl-1H-inden-4-yl)-3-pyridinecarboxamide; 3,4,5-trichloro-2,6-pyridinedicarbonitrile; actinovate; cis-1-(4-chlorophenyl)-2-(1H-1,2,4-triazol-1-yl)cycloheptanol; methyl 1-(2,3-dihydro-2,2-dimethyl-1H-inden-1-yl)-1H-imidazole-5-carboxylate; monopotassium carbonate; N-(6-methoxy-3 -pyridinyl)cyclopropanecarboxamide; sodium tetrathiocarbonate; and copper salts and preparations, such as Bordeaux mixture; copper hydroxide; copper naphthenate; copper oxychloride; copper sulphate; cufraneb; copper oxide; mancopper; oxine-copper.

Bactericides:
bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.

Insecticides/acaricides/nematicides:
abamectin, ABG-9008, acephate, acequinocyl, acetamiprid, acetoprole, acrinathrin, AKD-1022, AKD-3059, AKD-3088, alanycarb, aldicarb, aldoxycarb, allethrin, allethrin 1R-isomers, alpha-cypermethrin (alphamethrin), amidoflumet, aminocarb, amitraz, avermectin, AZ-60541, azadirachtin, azamethiphos, azinphos-methyl, azinphos-ethyl, azocyclotin, Bacillus popilliae, Bacillus sphaericus, Bacillus subtilis, Bacillus thuringiensis, Bacillus thuringiensis strain EG-2348, Bacillus thuringiensis strain GC-91, Bacillus thuringiensis strain NCTC-11821, baculoviruses, Beauveria bassiana, Beauveria tenella, bendiocarb, benfuracarb, bensultap, benzoximate, beta-cyfluthrin, beta-cypermethrin, bifenazate, bifenthrin, binapacryl, bioallethrin, bioallethrin-S-cyclopentyl-isomer, bioethanomethrin, biopermethrin, bioresmethrin, bistrifluron, BPMC, brofenprox, bromophos-ethyl, bromopropylate, bromfenvinfos (-methyl), BTG-504, BTG-505, bufencarb, buprofezin, butathiofos, butocarboxim, butoxycarboxim, butylpyridaben, cadusafos, camphechlor, carbaryl, carbofuran, carbophenothion, carbosulfan, cartap, CGA-50439, chinomethionat, chlordane, chlordimeform, chloethocarb, chlorethoxyfos, chlorfenapyr, chlorfenvinphos, chlorfluazuron, chlormephos, chlorobenzilate, chloropicrin, chlorproxyfen, chlorpyrifos-methyl, chlorpyrifos (-ethyl), chlovaporthrin, chromafenozide, cis-cypermethrin, cis-resmethrin, cis-permethrin, clocythrin, cloethocarb, clofentezine, clothianidin, clothiazoben, codlemone, coumaphos, cyanofenphos, cyanophos, cycloprene, cycloprothrin, Cydia pomonella, cyfluthrin, cyhalothrin, cyhexatin, cypermethrin, cyphenothrin (1R-trans-isomer), cyromazine, DDT, deltamethrin, demeton-S-methyl, demeton-S-methylsulfone, diafenthiuron, dialifos, diazinon, dichlofenthion, dichlorvos, dicofol, dicrotophos, dicyclanil, diflubenzuron, dimethoate, dimethylvinphos, dinobuton, dinocap, dinotefuran, diofenolan, disulfoton, docusat-sodium, dofenapyn, DOWCO-439, eflusilanate, emamectin, emamectin-benzoate, empenthrin (1R-isomer), endosulfan, Entomopthora spp., EPN, esfenvalerate, ethiofencarb, ethiprole, ethion, ethoprophos, etofenprox, etoxazole, etrimfos, famphur, fenamiphos, fenazaquin, fenbutatin oxide, fenfluthrin, fenitrothion, fenobucarb, fenothiocarb, fenoxacrim, fenoxycarb, fenpropathrin, fenpyrad, fenpyrithrin, fenpyroximate, fensulfothion, fenthion, fentrifanil, fenvalerate, fipronil, flonicamid, fluacrypyrim, fluazuron, flubenzimine, flubrocythrinate, flucycloxuron, flucythrinate, flufenerim, flufenoxuron, flufenprox, flumethrin, flupyrazofos, flutenzin (flufenzine), fluvalinate, fonofos, formetanate, formothion, fosmethilan, fosthiazate, fubfenprox (fluproxyfen), furathiocarb, gamma-HCH, gossyplure, grandlure, granulosis viruses, halfenprox, halofenozide, HCH, HCN-801, heptenophos, hexaflumuron, hexythiazox, hydramethylnone, hydroprene, IKA-2002, imidacloprid, imiprothrin, indoxacarb, iodofenphos, iprobenfos, isazofos, isofenphos, isoprocarb, isoxathion, ivermectin, japonilure, kadethrin, nuclear polyhedrosis viruses, kinoprene, lambda-cyhalothrin, lindane, lufenuron, malathion, mecarbaam, mesulfenfos, metaldehyde, metam-sodium, methacrifos, methamidophos, Metharhizium anisopliae, Metharhizium flavoviride, methidathion, methiocarb, methomyl, methoprene, methoxychlor, methoxyfenozide, metolcarb, metoxadiazone, mevinphos, milbemectin, milbemycin, MKI-245, MON-45700, monocrotophos, moxidectin, MTI-800, naled, NC-104, NC-170, NC-184, NC-194, NC-196, niclosamide, nicotine, nitenpyram, nithiazine, NNI-0001, NNI-0101, NNI-0250, NNI-9768, novaluron, noviflumuron, OK-5101, OK-5201, OK-9601, OK-9602, OK-9701, OK-9802, omethoate, oxamyl, oxydemeton-methyl, Paecilomyces fumosoroseus, parathion-methyl, parathion (-ethyl), permethrin (cis-, trans-), petroleum, PH-6045, phenothrin (1R-trans isomer), phenthoate, phorate, phosalone, phosmet, phosphamidon, phosphocarb, phoxim, piperonyl butoxide, pirimicarb, pirimiphos-methyl, pirimiphos-ethyl, prallethrin, profenofos, promecarb, propaphos, propargite, propetamphos, propoxur, prothiofos, prothoate, protrifenbute, pymetrozine, pyraclofos, pyresmethrin, pyrethrum, pyridaben, pyridalyl, pyridaphenthion, pyridathion, pyrimidifen, pyriproxyfen, quinalphos, resmethrin, RH-5849, ribavirin, RU-12457, RU-15525, S-421, S-1833, salithion, sebufos, SI-0009, silafluofen, spinosad, spirodiclofen, spiromesifen, sulfluramid, sulfotep, sulprofos, SZI-121, tau-fluvalinate, tebufenozide, tebufenpyrad, tebupirimfos, teflubenzuron, tefluthrin, temephos, temivinphos, terbam, terbufos, tetrachlorvinphos, tetradifon, tetramethrin, tetramethrin (1R-isomer), tetrasul, theta-cypermethrin, thiacloprid, thiamethoxan, thiapronil, thiatriphos, thiocyclam hydrogenoxalate, thiodicarb, thiofanox, thiometon, thiosultap-sodium, thuringiensin, tolfenpyrad, tralocythrin, tralomethrin, transfluthrin, triarathene, triazamate, triazophos, triazuron, trichlophenidine, trichlorfon, triflumuron, trimethacarb, vamidothion, vaniliprole, verbutin, Verticillium lecanii, WL-108477, WL-40027, YI-5201, YI-5301, YI-5302, XMC, xylylcarb, ZA-3274, zeta-cypermethrin, zolaprofos, ZXI-8901, the compound 3-methylphenyl propylcarbamate (Tsumacide Z), the compound 3-(5-chloro-3-pyridinyl)-8-(2,2,2-trifluoroethyl)-8-azabicyclo[3.2.1]octane-3-carbonitrile (CAS-Reg. No. 185982-80-3) and the corresponding 3-endo-isomer (CAS-Reg. No. 185984-60-5) (cf. WO 96/37494, WO 98/25923), and preparations which comprise insecticidally active plant extracts, nematodes, fungi or viruses.

A mixture with other known active compounds, such as herbicides, or with fertilizers and growth regulators, safener and/or semiochemicals is also possible.

In addition, the compounds of the formula (I) according to the invention also have very good antimycotic activity. They have a very broad antimycotic activity spectrum in particular against dermatophytes and yeasts, molds and diphasic fungi (for example against Candida species such as Candida albicans, Candida glabrata) and Epidermophyton floccosum, Aspergillus species such as Aspergillus niger and Aspergillus fumigatus, Trichophyton species such as Trichophyton mentagrophytes, Microsporon species such as Microsporon canis and audouinii. The list of these fungi by no means limits the mycotic spectrum which can be covered, but is only for illustration.

The active compounds can be used as such, in the form of their formulations or the use forms prepared therefrom, such as ready-to-use solutions, suspensions, wettable powders, pastes, soluble powders, dusts and granules. Application is carried out in a customary manner, for example by watering, spraying, atomizing, broadcasting, dusting, foaming, spreading, etc. It is furthermore possible to apply the active compounds by the ultra-low volume method, or to inject the active compound preparation or the active compound itself into the soil. It is also possible to treat the seeds of the plants.

When using the active compounds according to the invention as fungicides, the application rates can be varied within a relatively wide range, depending on the kind of application. For the treatment of parts of plants, the active compound application rates are generally between 0.1 and 10 000 g/ha, preferably between 10 and 1000 g/ha. For seed dressing, the active compound application rates are generally between 0.001 and 50 g per kilogram of seed, preferably between 0.01 and 10 g per kilogram of seed. For the treatment of the soil, the active compound application rates are generally between 0.1 and 10 000 g/ha, preferably between 1 and 5000 g/ha.

As already mentioned above, it is possible to treat all plants and their parts according to the invention. In a preferred embodiment, wild plant species and plant cultivars, or those obtained by conventional biological breeding, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof, are treated. The term “parts” or “parts of plants” or “plant parts” has been explained above.

Particularly preferably, plants of the plant cultivars which are in each case commercially available or in use are treated according to the invention. Plant cultivars are to be understood as meaning plants having new properties (“traits”) and which have been obtained by conventional breeding, by mutagenesis or by recombinant DNA techniques. They can be cultivars, varieties, bio- or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The transgenic plants or plant cultivars (i.e. those obtained by genetic engineering) which are preferably to be treated according to the invention include all plants which, in the genetic modification, received genetic material which imparts particularly advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defense of the plants against animal and microbial pests, such as against insects, mites, phytopathogenic fungi, bacteria and/or viruses, and also increased tolerance of the plants to certain herbicidal active compounds. Examples of transgenic plants which may be mentioned are the important crop plants, such as cereals (wheat, rice), maize, soya, potatoes, cotton, tobacco, oilseed rape and also fruit plants (with the fruits apples, pears, citrus fruits and grapes), and particular emphasis is given to maize, soya, potatoes, cotton, tobacco and oilseed rape. Traits that are emphasized are in particular increased defense of the plants against insects, arachnids, nematodes and slugs and snails by toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (hereinbelow referred to as “Bt plants”). Traits that are also particularly emphasized are the increased defense of the plants against fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulphonylureas, glyphosates or phosphinotricin (for example the “PAT” gene). The genes which impart the desired traits in question can also be present in combination with one another in the transgenic plants. Examples of “Bt plants” which may be mentioned are maize varieties, cotton varieties, soya varieties and potato varieties which are sold under the trade names YIELD GARD® (for example maize, cotton, soya), KnockOut® (for example maize), StarLink® (for example maize), Bollgard® (cotton), Nucoton® (cotton) and NewLeaf® (potato). Examples of herbicide-tolerant plants which may be mentioned are maize varieties, cotton varieties and soya varieties which are sold under the trade names Roundup Ready® (tolerance to glyphosate, for example maize, cotton, soya), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned also include the varieties sold under the name Clearfield® (for example maize). Of course, these statements also apply to plant cultivars which have these genetic traits or genetic traits still to be developed, and which will be developed and/or marketed in the future.

The plants listed can be treated according to the invention in a particularly advantageous manner with the compounds of the general formula (I) or the active compound mixtures according to the invention. The preferred ranges stated above for the active compounds or mixtures also apply to the treatment of these plants. Particular emphasis is given to the treatment of plants with the compounds or mixtures specifically mentioned in the present text.

The preparation and the use of the active compounds according to the invention is illustrated by the examples below.

### EXAMPLES

### Preparation Examples

### Example 1 (Process a))

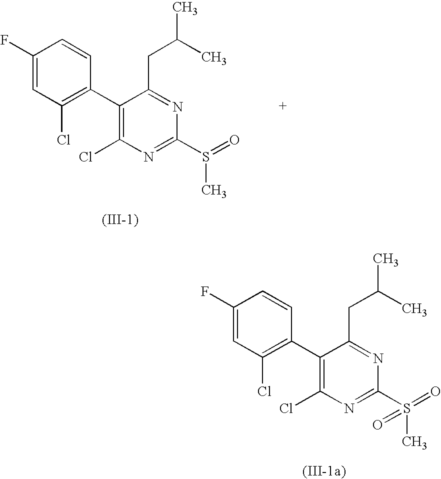

4.7 g (0.014 mol) of 4-chloro-5-(2-chloro-4-fluorophenyl)-6-isobutyl-2-(methylthio)pyrimidine are initially charged in 47 ml of dichloromethane and mixed with 1 ml (0.027 mol) of formic acid and 0.67 g (0.003 mol) of ammonium molybdate. At room temperature, 3.6 ml of a 35% strength hydrogen peroxide solution are then added dropwise over a period of 20 minutes. The mixture is stirred at room temperature for a further 12 hours. 10 ml of water are then added, and the organic phase is separated off. The organic phase is washed with 10 ml of dilute sodium hydrogensulphite solution, dried over sodium sulphate and then concentrated under reduced pressure. The residue is then chromatographed in a mixture of n-hexane:ethyl acetate=gradient from 3:1 to 1:1 on silica gel.

1.7 g of 4-chloro-5-(2-chloro-4-fluorophenyl)-6-isobutyl-2-(methylsulphinyl)pyrimidine (logP=3.27; content by HPLC: 92%) are obtained as first fraction and 1.9 g of 4-chloro-5-(2-chloro-4-fluorophenyl)-6-isobutyl-2-(methylsulphonyl)pyrimidine (logP=3.82; content by HPLC: 73%) are obtained as second fraction.

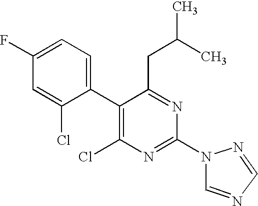

At room temperature, 0.5 g (0.0014 mol) of 4-chloro-5-(2-chloro-4-fluorophenyl)-6-isobutyl-2-(methylsulphinyl)pyrimidine in 10 ml of N,N-dimethylformamide is mixed with 0.096 g (0.0014 mol) of 1H-1,2,4-triazole and 0.2 ml (0.0015 mol) of triethylamine. The mixture is then stirred at 60° C. for 12 hours. The reaction mixture is concentrated under reduced pressure. 10 mol of dilute 1N hydrochloric acid and 10 ml of dichloromethane are then added to the residue. The organic phase is dried over sodium sulphate and then concentrated under reduced pressure. The residue is chromatographed in a mixture of n-hexane:ethyl acetate=5:1 on silica gel. This gives 0.18 g of 4-chloro-5-(2-chloro-4-fluorophenyl)-6-isobutyl-2-(1H-1,2,4-triazol-1-yl)pyrimidine (logP=3.79; content by HPLC: 98%).

Analogously to Example 1 the following intermediates of formulae (II) and (III) were obtained:

| Ex. | | | | | | | | | |
|---|---|---|---|---|---|---|---|---|---|
| No. | R¹ | R² | R³ | R⁴ | R⁵ | R⁶ | R⁷ | R⁸ | logP |
| 2 | i-Butyl | —S—CH₃ | —Cl | —Cl | —H | —H | —H | —F | 5.66 |
| II-2 | i-Butyl | —S—CH₃ | —Cl | —H | —H | —F | —H | —Cl | 5.67 |
| III-1 | i-Butyl | —SO—CH₃ | —Cl | —Cl | —H | —H | —H | —F | 3.08 |
| III-1a | i-Butyl | —SO₂—Me | —Cl | —Cl | —H | —H | —H | —F | 3.59 |
| III-2a | i-Butyl | —SO₂—Me | —Cl | —H | —H | —F | —H | —Cl | 3.82 |
| III-3a | i-Butyl | —SO₂—Me | i-Butyl | —H | —H | —F | —H | —Cl | 4.31 |

### Example 2 (Process b))

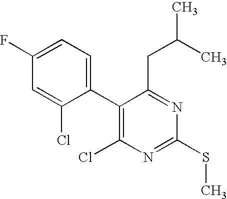

Under argon, 5.9 g (0.018 mol) of 4,6-dichloro-5-(2-chloro-4-fluorophenyl)-2-(methylthio)pyrimidine and 0.32 g (0.0009 mol) of iron(III) acetylacetonate are initially charged in a mixture of 30 ml of tetrahydrofuran and 2.8 ml of N-methylpyrrolidone. At room temperature, 8.8 g (0.055 mol) of isobutylmagnesium bromide are added. The mixture is stirred at room temperature for 1 hour. With cooling, the mixture is then slowly poured into a mixture of 1N hydrochloric acid (15 ml) and ethyl acetate (15 ml). After phase separation, the aqueous phase is extracted with further ethyl acetate. The combined organic phases are dried over sodium sulphate and concentrated under reduced pressure. The residue is then chromatographed in a mixture of petroleum ether:tert-butyl methyl ether=100:1 on silica gel.

This gives 4.7 g of 4-chloro-5-(2-chloro-4-fluorophenyl)-6-isobutyl-2-(methylthio)pyrimidine (logP=5.67; content by HPLC: 69%).

Analogously to Examples 1 and 2 and in accordance with the general statements in the general descriptions of processes a) to d), it is also possible to obtain the compounds of the formula (I) listed in Table 41 below.

TABLE 41

| | |
|---|---|
| | (I) |
| 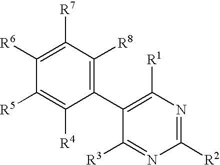 | |

| Ex. | | | | | | | | | |
|---|---|---|---|---|---|---|---|---|---|
| No. | R¹ | R² | R³ | R⁴ | R⁵ | R⁶ | R⁷ | R⁸ | logP |
| 3 | CH₂CH(CH₃)₂ | 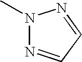 | Cl | Cl | H | H | H | F | 3.75 |
| 4 | CH₂CH(CH₃)₂ | 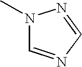 | Cl | Cl | H | H | H | F | 3.59 |
| 5 | CH₂CH(CH₃)₂ | 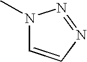 | Cl | Cl | H | H | H | F | 3.84 |
| 6 | CH₂CH(CH₃)₂ | 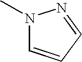 | Cl | H | H | F | H | Cl | 4.6 |
| 7 | CH₂CH(CH₃)₂ | 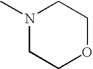 | Cl | H | H | F | H | Cl | 5.58 |
| 8 | CH₂CH(CH₃)₂ | 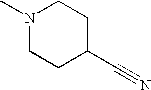 | Cl | H | H | F | H | Cl | 5.47 |
| 9 | —CH₂CH₂CH₃ | 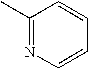 | Cl | H | H | H | H | H | 2.29 |
| 10 | —CH₂—CH(CH₃)₂ | 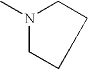 | Cl | Cl | H | F | H | H | 5.72 |
| 11 | —CH₂—CH(CH₃)₂ | 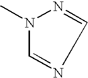 | CH₂—CH(CH₃)₂ | Cl | H | F | H | H | 4.77 |

The logP values were determined in accordance with EEC Directive 79/831 Annex V. A8 by HPLC (gradient method, acetonitrile/0.1% aqueous phosphoric acid).

### Use Examples

### Example A

Podosphaera Test (Apple)/Protective

Solvent: 24.5 parts by weight of acetone
24.5 parts by weight of dimethylacetamide

Emulsifier: 1 part by weight of alkylarylpolyglycol ether

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are inoculated with an aqueous spore suspension of the apple mildew pathogen Podosphaera leucotricha. The plants are then placed in a greenhouse at about 23° C. and a relative atmospheric humidity of about 70%.

Evaluation is carried out 10 days after the inoculation. 0% means an efficacy which corresponds to that of the control, whereas an efficacy of 100% means that no infection is observed.

In this test, the compounds according to the invention, at an application rate of 100 g/ha, show very high efficacy.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

1. A compound of the formula (I) 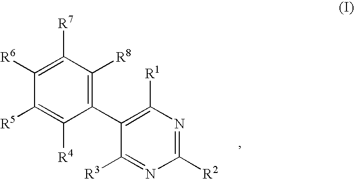
in which
R¹ represents C₁-C₈-alkyl, C₂-C₈-alkenyl, C₂-C₈-alkynyl, C₃-C₈-cycloalkyl, C₃-C₈-cycloalkenyl or represents a three- to ten-membered saturated mono- or bicyclic heterocycle which is attached via carbon and contains one to four heteroatoms from the group consisting of O, N and S, where R¹ may be substituted by one to three identical or different groups R^{a} and
R^{a} represents halogen, alkylamino, alkylhydrazino, cyano, oxo, nitro, C₁-C₆-alkylthio, C₁-C₆-alkyl, C₁-C₆-haloalkyl, tri(C₁-C₄-alkyl)silyl and/or C₁-C₆-alkoxy and/or represents unsubstituted or halogen-, C₁-C₄-alkyl- or C₁-C₄-haloalkyl-substituted C₃-C₆-cycloalkyl,
R² represents a three- to ten-membered saturated, partially unsaturated or aromatic mono- or bicyclic heterocycle which contains one to four heteroatoms from the group consisting of O, N and S, where R² may be substituted by one to three identical or different groups R^{b}, and
R^{b} represents halogen, hydroxyl, cyano, oxo, nitro, amino, mercapto, C₁-C₆-alkyl, C₁-C₆-haloalkyl, C₂-C₆-alkenyl, C₂-C₆-alkynyl, C₃-C₆-cycloalkyl, C₁-C₆-alkoxy, C₁-C₆-haloalkoxy, carboxyl, C₁-C₇-alkoxycarbonyl, carbamoyl, C₁-C₇-alkylaminocarbonyl, C₁-C₆-alkyl-C₁-C₆-alkylaminocarbonyl, morpholinocarbonyl, pyrrolidinocarbonyl, C₁-C₇-alkylcarbonylamino, C₁-C₆-alkylamino, di-(C₁-C₆-alkyl)amino, C₁-C₆-alkylthio, C₁-C₆-alkylsulphinyl, C₁-C₆-alkylsulphonyl, hydroxysulphonyl, aminosulphonyl, C₁-C₆-alkylaminosulphonyl and/or di-(C₁-C₆-alkyl)aminosulphonyl;
R³ represents halogen or C₁-C₈-alkyl, C₁-C₈-alkoxy, C₁-C₈-haloalkyl, C₁-C₈-alkylthio, C₁-C₈-alkylsulphinyl, C₁-C₈-alkylsulphonyl, or cyano,
R⁴,R⁸ independently of one another represent hydrogen, halogen, C₁-C₆-alkyl, C₁-C₆-haloalkyl or C₁-C₆-alkoxy,
R⁵, R⁷ independently of one another represent hydrogen, halogen, C₁-C₆-alkyl, C₁-C₆-haloalkyl, unsubstituted or halogen- or C₁-C₆-alkyl-substituted C₃-C₆-cycloalkyl, phenyl, phenoxy or phenylthio, where R⁵ and R⁷, when they represent phenyl, phenoxy or phenylthio, may be substituted by one to three identical or different groups R^{c}, and
R^{c} represents halogen, cyano, nitro, C₁-C₆-alkyl, C₁-C₆-haloalkyl, C₁-C₆-alkoxy and/or C₃-C₆-cycloalkyl,
R⁶ represents hydrogen, halogen, cyano, nitro, C₁-C₆-alkyl, C₁-C₆-haloalkyl, C₁-C₆-alkoxy, C₃-C₆-cycloalkoxy, C₁-C₆-haloalkoxy, C₁-C₆-alkoxycarbonyl, C₁-C₆-haloalkoxycarbonyl, unsubstituted or halogen- or C₁-C₆-alkyl-substituted C₃-C₆-cycloalkyl, aminocarbonyl, C₁-C₆-alkylcarbonyl, C₁-C₆-alkylaminocarbonyl, C₁-C₆-dialkylaminocarbonyl, C₁-C₆-alkylthio; C₁-C₆-alkylsulphinyl; C₁-C₆-alkylsulphonyl, alkylaminosulphonyl or dialkylaminosulphonyl or phenyl, phenoxy, phenylthio, where R⁶, when it represents phenyl, phenoxy or phenylthio, may be substituted by one to three identical or different groups R^{d} and
R^{d} represents halogen, cyano, nitro, C₁-C₆-alkyl, C₁-C₆-haloalkyl, C₁-C₆-alkoxy and/or C₃-C₆-cycloalkyl.

2. A compound of the formula (I) according to claim 1 in which
R¹ represents C₁-C₆-alkyl, C₂-C₆-alkenyl, C₃-C₆-alkynyl, C₃-C₆-cycloalkyl or represents a three- to ten-membered saturated mono- or bicyclic heterocycle which is attached via carbon, where R¹ may be substituted by one to three identical or different groups R^{a} and
R^{a} represents halogen, alkylamino, alkylhydrazino, cyano, oxo, nitro, C₁-C₄-alkylthio, C₁-C₄-alkyl, C₁-C₄-haloalkyl, trimethylsilyl and/or C₁-C₄-alkoxy and/or unsubstituted or halogen-, C₁-C₄-alkyl or C₁-C₄-haloalkyl-substituted C₃-C₆-cycloalkyl.

3. A compound according to claim 1 in which
R¹ represents a radical of the formula 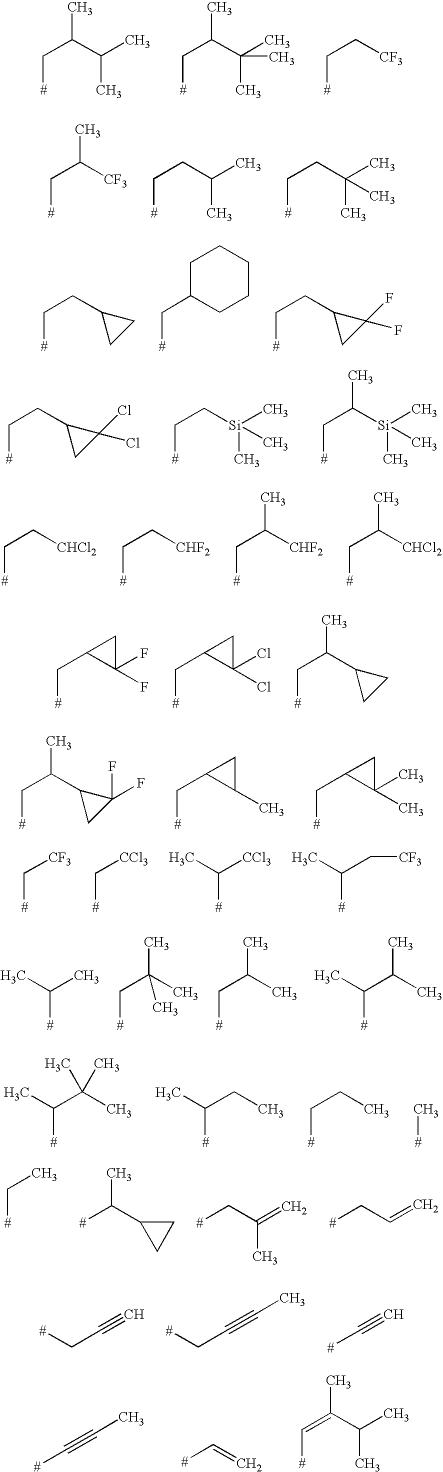 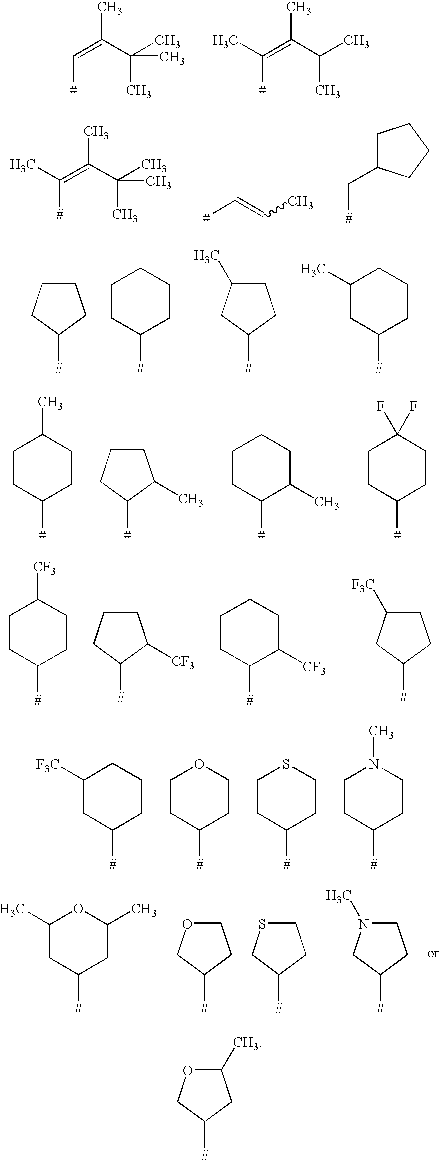
where # marks in each case the point of attachment.

4. A compound according to claim 1 in which R² is a three-, five or six-membered heterocycle.

5. A compound according to claim 1 in which
R² represents pyrrole, pyrazole, imidazole, 1,2,4-triazole, 1,2,3-triazole, tetrazole, 1,2,3-triazine, 1,2,4-triazine, oxazole, isoxazole, 1,3,4-oxadiazole, 1,3,4-thiadiazole, furan, thiophene, thiazole or isothiazole, where the heterocycle may be attached to the pyrimidine ring via carbon or nitrogen, R² may be substituted by one to three identical or different groups R^{b}, and
R^{b} represents halogen, hydroxyl, cyano, nitro, amino, mercapto, C₁-C₄-alkyl, C₁-C₄-haloalkyl, C₂-C₄-alkenyl, C₂-C₆-alkynyl, C₃-C₆-cycloalkyl, C₁-C₄-alkoxy, C₁-C₄-haloalkoxy, carboxyl, C₁-C₄-alkoxycarbonyl, carbamoyl, C₁-C₇-alkylaminocarbonyl, C₁-C₆-alkyl-C₁-C₆-alkylaminocarbonyl, morpholinocarbonyl, pyrrolidinocarbonyl, C₁-C₄-alkylcarbonylamino, C₁-C₄-alkylamino, di-(C₁-C₄-alkyl)amino, C₁-C₄-alkylthio, C₁-C₄-alkylsulphinyl, C₁-C₄-alkylsulphonyl, hydroxysulphonyl, aminosulphonyl, C₁-C₄-alkylaminosulphonyl or di-(C₁-C₄-alkyl)aminosulphonyl.

6. A compound according to claim 1 in which R² represents pyrazole, pyrrole, imidazole, 1,2,3-triazole, 1,2,4-triazole, 1,3,4-oxadiazole, 1,3,4-thiadiazole, tetrazole, 2-pyridine, 2-pyrimidine, pyrazine or 3-pyridazine, in each case optionally substituted by up to three groups R^{b} defined as in claim 1.

7. A compound according to claim 1 in which R² represents pyrazole, 1,2,3-triazole or 1,2,4-triazole, each of which is unsubstituted or mono-substituted by halogen, cyano, nitro, methyl or methoxy.

8. A compound according to claim 1 in which R³ represents halogen, C₁-C₆-alkyl, C₁-C₆-haloalkyl or C₁-C₆-alkoxy.

9. A compound according to claim 1 in which R³ represents halogen.

10. A compound according to claim 1 in which R³ represents chlorine.

11. A compound according to claim 1 in which R⁴ and R⁸ are not both hydrogen.

12. A compound according to claim 1 in which R⁴ represents hydrogen.

13. A compound according to claim 1 in which R⁴ represents hydrogen and R⁸ represents halogen or methyl.

14. A compound according to claim 1 in which R⁶ represents hydrogen, halogen or C₁-C₄-alkoxy.

15. A composition for controlling harmful organisms comprising at least one compound as defined in claim 1 and extenders and/or carriers and, if appropriate, surfactants.

16. A method for controlling harmful organisms comprising allowing a compound as defined in claim 1 to act on harmful organisms and/or their habitat.

17. A method for controlling harmful organisms comprising allowing compositions as defined in claim 15 to act on harmful organisms and/or their habitat.

18. A compound of the formula (II) 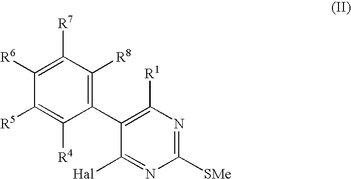
in which
R¹, R⁴, R⁵, R⁶, R⁷ and R⁸ are as defined in claim 1 and in which Hal represents halogen.

19. A process for preparing compounds of the formula (I) as defined in claim 1 in which R² represents a heterocycle attached via nitrogen and R³ represents halogen (compounds of the formula (I′)) comprising oxidizing compounds of the formula 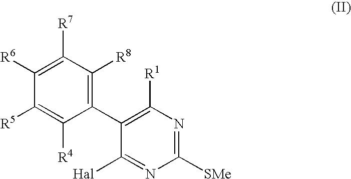
in which
R¹, R⁴, R⁵, R⁶, R⁷ and R⁸ are as defined in claim 1 and
Hal represents halogen,
using an oxidizing agent, if appropriate in the present of a diluent, to form a compound of the formula (III) 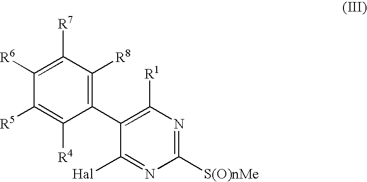
in which R¹, R⁴, R⁵, R⁶, R⁷, R⁸ and Hal are as defined in claim 1 and n is 1 or 2, and
reacting the compound of the formula (III) with a compound of the formula (IV)
R²—H (IV),
in which R² is as defined in claim 1, if appropriate in the presence of a diluent and if appropriate in the presence of an acid acceptor.

20. A process for preparing compounds of the formula (I) in which R² represents a heterocycle which is attached via carbon and R³ represents halogen (compounds of the formula (I″)) comprising reacting compounds of the formula (V) 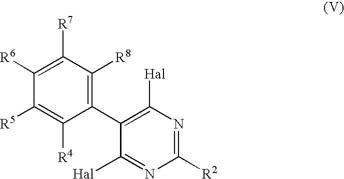
in which
R¹, R², R⁴, R⁵, R⁶, R⁷ and R⁸ are as defined in claim 1 and
Hal represents halogen,
with a compound of the formula (VI)
R¹-M¹ (VI),
in which
R¹ is as defined above and
M¹ represents lithium, sodium, potassium, dihydroxyboranyl, a radical of the formula 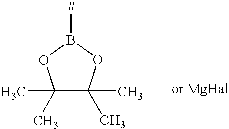
in which Hal represents chlorine, bromine or iodine,
if appropriate in the presence of a diluent and if appropriate in the presence of a catalyst.

21. A process for preparing compounds of the formula (I) in which R² represents a heterocycle which is attached via nitrogen or carbon and R³ represents C₁-C₈-alkyl, C₁-C₈-alkoxy, C₁-C₈-alkylthio, C₁-C₈-alkylsulphinyl, C₁-C₈-alkylsulphonyl or cyano (compounds of the formula (I′″)) comprising reacting compounds of the formula (I′) or (I″) 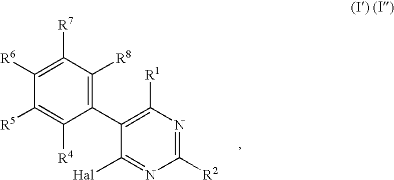
in which
R¹, R², R⁴, R⁵, R⁶, R⁷ and R⁸ are as defined in claim 1 and
Hal represents halogen,
either
(c1) with a compound of the formula
R³-M² (VII),
in which
R³ represents C₁-C₈-alkoxy, C₁-C₈-alkylthio, C₁-C₈-alkylsulphinyl, C₁-C₈-alkylsulphonyl or cyano, and
M² represents sodium or potassium,
if appropriate in the presence of a diluent,
or
(c2) with Grignard compounds of the formula
R³—Mg Hal (VIII)
in which
R³ represents C₁-C₈-alkyl,
Hal represents chlorine or bromine,
in the presence of a diluent and, if appropriate in the presence of a catalyst.

22. A process for preparing compounds of the formula (I) in which R² represents a heterocycle which is attached via nitrogen or carbon and R³ represents C₁-C₈-alkyl or C₁-C₈-haloalkyl (compounds of the formula (I′″)) comprising
(d1) reacting compounds of the formula (IX) 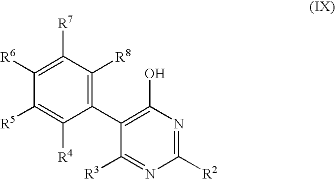
in which R², R³, R⁴, R⁵, R⁶, R⁷ and R⁸ are as defined in claim 1, with a halogenating agent, if appropriate in the presence of an acid acceptor and if appropriate in the presence of a diluent, to form compounds of the formula (X) 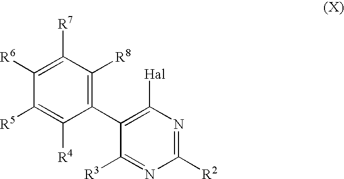
in which
R², R³, R⁴, R⁵, R⁶, R⁷ and R⁸ are as defined in claim 1 and
Hal represents halogen,
and
(d2) reacting the compounds of the formula (X) with a compound of the formula (VI)
R¹-M¹ (VI),
in which
R¹ is as defined above and
M¹ represents lithium, sodium, potassium, dihydroxyboranyl, a radical of the formula 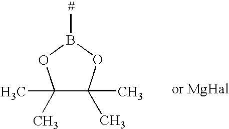
in which Hal represents chlorine, bromine or iodine,
if appropriate in the presence of a diluent and if appropriate in the presence of a catalyst.
